(19) United States
(12) Patent Application Publication
(10) Pub. No.: US 2025/0251360 A1
(43) Pub. Date: Aug. 7, 2025

(54) REDOX CAPACITANCE SENSING OF PARTICLES UNDER FLOW

(71) Applicant: Oxford University Innovation Limited, Oxford (GB)

(72) Inventors: Sophie Patrick, Oxford (GB); Robert Hein, Oxford (GB); Paul Beer, Oxford (GB); Jason Davis, Oxford (GB)

(21) Appl. No.: 18/706,602

(22) PCT Filed: Oct. 31, 2022
(86) PCT No.: PCT/GB2022/052748

§ 371 Date: May 1, 2024

(30) Foreign Application Priority Data
Nov. 2, 2021 (GB) 2115704.5

(51) Int. Cl. G01N 27/327; G01N 27/22

(52) U.S. Cl. CPC G01N 27/327; G01N 27/221

(57) ABSTRACT

The present invention relates to a technique for continuous sensing of particles, such as ions, under flow. The technique involves the use of continuous and polarisation-tuned redox capacitive sensing at suitably designed electroactive interfaces.

The present invention relates to the electrochemical sensing of particles under flow using redox capacitance measurements.

### BACKGROUND

Continuous, real-time sensing of particles, such as ions, is of great value across various environmental and medical scenarios, but remains underdeveloped.

To date, the only generically and widely applicable ion sensors are ion-selective electrodes. These are cheap, simple, and can also, in principle, be used for the continuous sensing of ions. However, they often suffer from comparably slow response times and thermodynamically limited sensitivities, which is problematic when real-time continuous monitoring of ions is sought, as would be desired, for example, in the monitoring of (waste) water streams.

Due to these limitations other electroanalytical methodologies for particle sensing are being developed, most notably voltammetric sensing using redox-active receptors (see, e.g., Hein et al., “Electrochemical Anion Sensing: Supramolecular Approaches” Chem. Rev. 2020, 120, 1888-1935). While academically well-established, electrochemical approaches have not been utilized in real-life sensing scenarios, mainly because they cannot generally be utilised in a simple, continuous signal-generating fashion.

As such, there remains a need to enable facile continuous, real-time particle sensing at redox-active receptive interfaces.

### SUMMARY OF THE INVENTION

The present inventors have developed a new technique for continuous sensing of particles, including ions, under flow. The technique involves the use of continuous and polarisation-tuned redox capacitive sensing at suitably designed electroactive interfaces.

In particular, the redox activity of an electroactive receptive interface can be accurately measured by redox capacitance spectroscopy, affording analogous information to that typically resolved by voltammetric methods of the like previously proposed. Specifically, the so-called redox density of states (DOS) distribution is directly dependent on the ratio of oxidized and reduced receptor states on the electroactive interface. Upon binding of particles (e.g., ions) to receptors states, this redox distribution shifts, thereby reporting on particle presence, as previously measured voltammetrically.

However, the inventors have found that monitoring this shift by redox capacitance DOS measurements, rather than voltammetrically, is associated with a number of unique advantages. This is enabled by the “at-equilibrium” nature of the redox capacitance measurements: in contrast to voltammetry, which measures the redox properties by repeatedly altering the surface potential, the redox capacitance measurements can be carried out continuously and at any chosen individual point of the DOS distribution, i.e. at a broad range of specific surface potentials. This single point (fixed potential) redox capacitance measurement enables:
target particle sensing with a simple, continuous, direct readout (not possible via voltammetry)
target particle sensing with a higher sensitivity than when using standard voltammetry
target particle sensing at any chosen electrode potential (not possible with any other electroanalytical technique)—this is further associated with a unique possibility to tune various sensing parameters, depending on the electrode potential:
   modulation of signal magnitude and “switch-on” or “switch-off” assaying
   tuning of target particle binding affinities and sensitivities: at any chosen potential the ratio of oxidized and reduced receptor states is fixed and determines the binding affinity
   in comparison to voltammetry, fixing the electrode potential also leads to a unique binding and signal self-amplification mechanism which enhances analytical performance (not possible with any other electroanalytical technique).
improved temporal resolution of changes in particle concentration in the fluid flowing past the electrode compared with voltammetric methods.

The methodology thus enables facile, continuous and real-time sensing of any analyte (including anions, cations, and neutrally charged molecules and atoms) which can reversibly perturb the voltammetric profile of a receptive, redox-active interface.

The present invention thus provides an electrochemical method of sensing target particles in a carrier medium under flow, which method comprises:
(A) providing a flow path that comprises an electrode, the electrode being positioned to contact the carrier medium when the carrier medium flows along the flow path, and the electrode comprising redox-active receptors capable of binding to the target particles to change the redox capacitance of the electrode;
(B) flowing the carrier medium along the flow path over a flow period, such that a continuously changing portion of the carrier medium contacts the electrode throughout the flow period; and
(C) continuously obtaining redox capacitance measurements of the electrode over the flow period, thereby sensing the binding of target particles to the redox-active receptors and hence the presence, or absence, of target particles in the portion of carrier medium that is in contact with the electrode at each time point over the flow period.

Further preferred features and embodiments are described in the accompanying description and the appended claims.

### DESCRIPTION OF THE DRAWINGS

FIG. 1 shows A) Schematic representation of 1.XB/HB_{SAM} on a gold electrode. B) CV and SWV of 1.XB_{SAM} in ACN/H₂O 99:1, 100 mM TBAClO₄, 10 mM HClO₄. C) Capacitive Nyquist plots of 1.XB_{SAM} in ACN/H₂O 99:1, 100 mM TBAClO₄, 10 mM HClO₄, at E_{1/2} (squares). Each data point represents a measurement at a different AC frequency. The film redox capacitance is then obtained as the real capacitance C′ at the frequency of the inflection point (fᵣ), approximately the semicircle diameter.

FIG. 2 shows: in Panel A, Capacitive Nyquist plots of 1.XB_{SAM} in ACN/H₂O 99:1, 100 mM TBAClO₄, 10 mM HClO₄, at E_{1/2} (squares) and E₋₂₄₀ (circles). Each data point represents a measurement at a different AC frequency. The film (redox) capacitance is then obtained as the real capacitance C′ at the frequency of the inflection point (fᵣ), which approximates the semicircle diameter for the Nyquist recorded at E_{1/2}; in Panel B, Raw, absolute redox distributions showing redox capacitance Cr of 1.XB_{SAM} in ACN/H₂O 99:1 (100 mM TBAClO₄, 10 mM HClO₄) as function of potential (density of states, DOS), in response to increasing concentrations of HSO₄⁻ up to 50 mM; and in Panel C, Voltammetric response isotherms obtained of 1.XB_{SAM} in ACN/H₂O 99:1 (100 mM TBAClO₄, 10 mM HClO₄), in response to increasing concentrations of HSO₄ up to 50 mM from standard voltammetric titrations by SWV (squares), as well as from the peak shift of the redox capacitance DOS distribution (circles) highlighting that both approaches afford the same voltammetric (potential response) information.

FIG. 3 shows a comparison of SWV (squares) and redox capacitance Cᵣ (triangles) as a function of electrode potential (i.e. redox density of states (DOS)) of 1.XB_{SAM} in ACN/H₂O 99:1, 100 mM TBAClO₄, 10 mM HClO₄. Cᵣ(E) was obtained at a fixed frequency fᵣ (determined at E_{1/2}, see FIG. 1C). Schematically depicted is the ratio of oxidised and reduced anion receptors at different potential regimes.

FIG. 4 shows response of 1.XB_{SAM} in ACN/H₂O 99:1, 100 mM TBAClO₄, 10 mM HClO₄, upon exposure to increasing concentrations of HSO₄⁻. Panel A: Normalised redox capacitance Cᵣ as function of potential (the film density of states, DOS). The DOS at each concentration was, for simplicity and illustrative purposes, normalised to the initial C_{E1/2} (i.e. to the initial peak redox capacitance). The raw, absolute redox distributions at each potential are shown in FIG. 2B. The solid arrow indicates the “standard” potential shift as typically resolved voltammetrically. The dashed arrow indicates the drop in Cᵣ at E_{1/2} (dotted line created by square data points). Panel B: Voltammetric response isotherms obtained from standard voltammetric titrations by SWV (squares). Shown as stars is the decrease in Cᵣ at E_{1/2} as a result of anion-induced cathodic shifting of the DOS such that C_{E1/2} lies in the increasingly smaller tail regime.

FIG. 5 has three panels A, B and C. Panel A shows continuous capacitance response of 1.XB_{SAM} under pseudo flow conditions in a stirred three-electrode cell. The trace with square data points depicts the sequential addition of aliquots of HSO₄⁻ up to a final concentration of 50 mM. The trace with circular data points depicts the response upon continuous addition of up to 50 mM HSO₄⁻ via a syringe pump (flow rate Q=0.8 mL min⁻¹, into an initial electrolyte volume of 4 mL). Note the different time frames (x-axis scaling) for both graphs. Panel B shows capacitance response of 1.XB_{SAM} under completely static conditions (squares) and under pseudo flow conditions in a stirred, but otherwise static, three-electrode cell at E_{1/2}. The circles depict the binding isotherm corresponding to the sequential addition of increasing concentrations of up to 50 mM HSO₄⁻. Shown in triangles is the continuous binding isotherm corresponding to the continuous addition of up to 50 mM HSO₄⁻ via a syringe pump (V_{Sample}=4 mL, flow rate Q=0.8 mL min⁻¹, into an initial electrolyte volume of 4 mL). The somewhat later onset of the response under continuous addition arises from a mixing delay. Panel C shows absolute (sub-panel A) and relative (sub-panel B) capacitance response of 1.XB_{SAM} towards HSO₄⁻ (up to a final concentration of 50 mM) at different electrode potentials, as determined by continuous, pseudo-flow experiments (continuous anion addition via syringe-pump into stirred cell). Note that the relative response in sub-panel B is normalised to the baseline capacitance at each individual potential and not the baseline capacitance at E_{1/2}.

FIG. 6 is a schematic depiction of the 3D printed electrochemical flow cell used in the examples.

FIG. 7 has three panels A, B and C. Panel A shows dependence of redox capacitance signal (measured at E_{1/2}) on flow rate, over a range of 300 to 2000 μL min⁻¹, with sub-panel A scaled to a range encompassing a typical capacitive response towards anion binding and sub-panel B and over a narrower range (zoomed in). Panel B shows redox capacitance response of 1.XB_{SAM} towards blank samples (running electrolyte, 100 mM TBAClO₄), control samples (25 mM TBAPF₆), 0.5 mM HSO₄⁻ and 25 mM HSO₄⁻ under continuous electrolyte flow (flow rate Q=500 μL min⁻¹, E=E_{1/2}, f=fᵣ). Each spike represents the response towards aliquots (V_{Sample}=0.5 mL) of the blank, control or HSO₄⁻ samples, as indicated. Panel C shows Redox capacitance response of 1.XB_{SAM} towards HSO₄⁻ under continuous electrolyte flow (flow rate Q=200 μL min⁻¹, E=E₋₂₀₀, f=fᵣ) over 4 h. Each spike in represents the response towards aliquots (V_{Sample}=0.5 mL) of 20 mM HSO₄⁻.

FIG. 8 shows redox capacitance response of 1.XB_{SAM} towards HSO₄⁻ under continuous electrolyte flow in the custom flow cell (cell volume=100 μL, flow rate Q=500 μL min⁻¹, E=E_{1/2}, f=fᵣ). Each spike represents the response towards aliquots (V_{Sample}=0.5 mL) of HSO₄⁻ of increasing concentrations up to 50 mM. The response isotherm was obtained by normalizing the peak response to the baseline before each addition. The solid line represents a fit according to the Langmuir-Freundlich model (Eqn. 1). Error bars represent one standard deviation of five independent experiments.

FIG. 9 shows, in panel A), redox capacitance response of 1.XB_{SAM} towards HSO₄⁻ at E_{1/2}, E₋₁₀₅, E₋₂₀₀ and E₊₁₀₀ under continuous electrolyte flow in a custom flow cell (cell volume=100 μL, flow rate Q=500 μL min⁻¹). Each spike in A) represents the response towards aliquots (V_{Sample}=0.5 mL) of HSO₄⁻ of increasing concentrations up to 50 mM with absolute signal increasing or decreasing depending on the initial surface polarisation. In panel B): the corresponding baseline-corrected response isotherms. Solid and dashed lines represent fits according to the Langmuir-Freundlich model (Eqn. 1)). The dashed black line represents, for simpler comparison, the mirrored capacitance response at E_{1/2}, highlighting the steeper response slope and enhanced anion binding magnitude at E_{1/2} vs at E₋₁₀₅. Error bars represent one standard deviation from at least three independent experiments. Shaded areas represent the 95% confidence interval. A more detailed comparison of the sensor responses are also shown in FIGS. 12A and 12B.

FIG. 10 shows theoretical binding constant K_{Theo} (trace having higher minimum and lower maximum) and percentage of oxidized receptor (trace having the lower minimum and higher maximum) of 1.XB_{SAM} towards HSO₄⁻ as a function of the electrode potential with respect to E_{1/2} as calculated via Eqn S7.15 and the Nernst equation.

FIG. 11 shows a schematic depiction of the anion-binding induced cathodic shift of the redox distribution. The circle depicts the decrease in measured redox capacitance at the constantly applied potential (E_{1/2}). Schematically illustrated underneath is the associated ratio of oxidised: neutral (reduced) receptors highlighting the anion binding-induced “oxidation” of the interface, with a concomitantly increased overall anion binding strength (“self-amplification”). It is worth noting that anion binding in the neutral state is negligible, and strong in the cationic state, see also FIG. 10.

FIG. 12 shows: in panel A, baseline-corrected response isotherms corresponding to the sensograms in FIG. 9 showing the response of 1.XB_{SAM} to HSO₄⁻ (up to 50 mM) as the change in capacitance. Solid lines represent fits according to the Langmuir-Freundlich model (Eqn. 1)). Error bars represent one standard deviation from at least three independent experiments; and in Panel B, baseline-corrected response isotherms corresponding to the sensograms in FIG. 9 showing the response of 1.XB_{SAM} to HSO₄⁻ (up to 50 mM) as the modulus of the change in capacitance. Solid lines represent fits according to the Langmuir-Freundlich model (Eqn. 1)). Error bars represent one standard deviation from at least three independent experiments.

FIG. 13 has three panels A, B and C. Panel A shows redox capacitance response of 1.XB_{SAM} towards H₂PO₄⁻ under continuous electrolyte flow in a custom flow cell, E=E_{1/2}. Each spike in represents the response towards aliquots of H₂PO₄⁻ of increasing concentrations up to 33 mM. The solid line represents a fit according to the Langmuir-Freundlich model (Eqn. 1). Error bars represent one standard deviation of three independent experiments. Panel B shows redox capacitance response of 1.XB_{SAM} towards H₂PO₄⁻ under continuous electrolyte flow, E=E₋₂₀₀. Each spike in represents the response towards aliquots of H₂PO₄⁻ of increasing concentrations up to 33 mM. The solid line represents a fit according to the Langmuir-Freundlich model (Eqn. 1). Error bars represent one standard deviation of three independent experiments. Panel C shows redox capacitance response isotherms of triplicate, independent repeats and their average response of 1.XB_{SAM} towards H₂PO₄⁻ at (sub-panel A) E_{1/2} and (sub-panel B) E₋₂₀₀ under continuous electrolyte flow. The response isotherms were corrected by-10 mM to account for the suppressed response below 10 mM due to full protonation of H₂PO₄. Solid lines represent fits according to the Langmuir-Freundlich model (Eqn. 1)). Error bars represent one standard deviation of three independent experiments.

FIG. 14 shows: in panel A, redox capacitance response of 1.XB_{SAM} towards Cl-under continuous electrolyte flow (ACN/H₂O 99:1) in a custom flow cell at E=E_{1/2}, V_{Sample}=1 mL. Each spike in represents the response towards aliquots of Cl of increasing concentrations up to 20 mM; in panel B, redox capacitance response of 1.XB_{SAM} towards Cl-under continuous electrolyte flow (ACN/H₂O 99:1) in a custom flow cell at E=E₋₂₀₀, V_{Sample}=1 mL. Each spike in represents the response towards aliquots of Cl-of increasing concentrations up to 20 mM.

FIG. 15 has three panels A, B and C. Panel A shows Redox capacitance response of 1.XB_{SAM} towards HSO₄⁻ at two potentials under continuous electrolyte flow, E=E₋₂₀₀ (lower trace) and E=E_{1/2} (upper trace). Each spike represents the response towards aliquots (V_{Sample}=0.5 mL) of HSO₄⁻ of increasing concentrations up to 50 mM. Panel B shows redox capacitance response of 1.XB_{SAM} towards HSO₄⁻ at two potentials under continuous electrolyte flow (sub-panel A) E=E_{1/2} and (sub-panel B) E=E₋₂₀₀. Each spike represents the response towards aliquots of HSO₄⁻ of increasing concentrations up to 50 mM for sensograms at (sub-panel A) E_{1/2} and (sub-panel B) E₋₂₀₀. The solid lines represent fits according to the Langmuir-Freundlich model (Eqn. 1). Panel C shows (sub-panel A) comparison of continuous capacitance response of 1.XB_{SAM} under pseudo flow conditions in a stirred, but otherwise static, three-electrode cell at E_{1/2} at fᵣ (4.2 Hz, squares) and 20.3 Hz (circles); (sub-panel B) the binding isotherms corresponding to the continuous addition of up to 50 mM HSO₄⁻ via a syringe pump (V_{Sample}=4 mL, flow rate Q=0.8 mL min⁻¹, into an initial electrolyte volume of 4 mL).

FIG. 16 shows, in panels (A) to (C) photos and in panel (D) schematic of 3D-printed microfluidic cell; and panel (E) a blueprint of 3D-printed microfluidic cell displaying all relevant dimensions in mm. R=radius, Ø=diameter.

### DETAILED DESCRIPTION

Optional and preferred features of the present disclosure are now described. Any of the features described herein may be combined with any of the other features described herein, unless otherwise stated.

The method of the invention utilises redox capacitance measurements as an electrochemical means for sensing the presence of target particles in a carrier medium flowing past a suitable designed electrode. The electrode, in particular, features redox-active receptors whose redox capacitance can be probed. The receptors are also capable of binding to the target particles. When bound to target particles, the redox capacitance characteristics of the electrode change. Typically, for instance, and as clearly demonstrated in the examples section, binding to target particles induces a shift in the half-wave potential (E_{1/2}) at which the redox capacitance is largest. Thus, for instance, measurement of the redox capacitance at a fixed potential (either at the half-wave potential or away from the half-wave potential of the electrode in the absence of target particles) changes if at least a portion of the receptors are bound to target particles. Furthermore, the magnitude of change in redox capacitance can be correlated to the concentration of bound target particles, and hence the concentration of target particles in the portion of the carrier fluid in contact with the electrode at the time of measurement of the redox capacitance. As explained already, and further outlined in detail in the examples section, sensing of target particles in this manner, utilising the modulations to redox capacitance caused by target particle to receptor binding on the electrode, gives rise to numerous advantages compared with previously developed sensors, as well as compared with sensing using an analogous electrode but applying voltammetric measurement techniques (e.g. square wave voltammetry).

### Carrier Medium and Target Particles

The methods of the present invention are widely applicable for assaying of particles in general. There is no particular limitation on the nature of target particles that can be sensed according to the invention, nor the carrier medium in which they are comprised.

In any particular embodiment of the invention, the combination of target particles and redox-active receptors comprised by the electrode is such that: (a) the target particles are capable of binding to the redox-active receptors; and (b) binding of the target particles to the redox-active receptors changes the redox capacitance of the electrode (e.g. by changing the half-wave potential).

The target particles may be either charged particles (referred to hereinafter as target ions) or neutrally charged particles. Preferably the target particles are target ions.

Examples of target ions include anions and cations. In one, preferred, embodiment of the invention, the target ions are target anions. In another embodiment, the target ions are target cations. The ions may be organic ions or inorganic ions. The ions may be monoatomic or polyatomic. For instance, the ions may be of interest as potential contaminants of a carrier medium.

Non-limiting specific examples of target ions include halide ions (e.g., Cl⁻, F⁻, Br⁻, I⁻), radioactive ions such as radioactive by-product ions of nuclear reactions including pertechnetates (e.g., TcO₄⁻), nitrates (e.g. NO₃⁻), organophosphates, phosphates (e.g., H₂PO₄⁻), such as nitrates, organophosphates and phosphates present in or derived from fertilizers, alkali metals (e.g., Na⁺, K⁺), and alkaline earth metals.

When the target particles are neutrally charged particles they may be neutrally charged molecules or neutrally charged atoms. One non-limiting example of such neutrally charged particles is sugar molecules.

The carrier medium is usually a fluid and most typically a liquid. The target particles, if present, may be dispersed and/or dissolved in the carrier medium (most commonly they are dissolved in the carrier medium). For instance, the carrier medium may comprise water or be an aqueous liquid, e.g. water. One exemplary practical use for the methods of the invention may be in the sensing of target particles in water such as waste water samples, e.g. where the target particles correspond to known potential contaminants of water supplies. However, the carrier medium can also be non-aqueous, e.g. a non-aqueous liquid.

The carrier medium may also be a biological fluid, e.g. in embodiments where the method is envisaged for use in a medical scenario. Non-limiting examples include blood and urine.

As will be clear to those skilled in the art, the carrier medium may, or may not, contain the target particles. When the carrier medium does contain the target particles, then they are sensed (e.g. their concentration determined) by performing the steps of the method of the invention. If the carrier medium, or a portion of the carrier medium, does not contain the target particles, then performing the steps of the method of the invention provides confirmation of their absence from the carrier medium as a whole, of from a portion thereof (i.e. because there has been no modulation to the redox capacitance characteristics of the electrode per se). Both scenarios fall within the definition of a method of sensing target particles of the invention.

Optionally, the carrier medium can be modified before or during the step (B) of flowing the carrier medium along the flow path (and contacting the electrode) in the method of the invention. For instance, the modification may comprising adding material (e.g. acid, supporting electrolyte, additional liquid) to the carrier medium prior to it flowing along the flow path. Alternatively, the modification may comprise adding material (e.g. acid, supporting electrolyte, additional liquid) to the carrier medium while it is flowing along the flow path (typically, though, prior to it contacting with the electrode). Material may be added either continuously or discontinuously (e.g. in one or more batches).

For instance, in some embodiments, it may be desirable to acidify the carrier medium in order to improve the stability of the electrode to the continuous redox capacitance measurements being obtained. One specific example is where the redox-active receptors comprise ferrocene, the oxidised ferrocenium (Fc⁺) portion of which is vulnerable to reaction with nucleophiles or electrolyte anions: this can be mitigated by acidification (e.g. with HClO₄), to thereby protonate the nucleophiles and scavenge them.

Furthermore, in some embodiments it may be desirable to add a supporting electrolyte to the carrier medium, for instance comprising large non-coordinating anions (like perchlorate (ClO₄⁻) or hexafluorophosphate (PF₆⁻), e.g., as tetrabutylammonium (TBA) salts). Such a supporting electrolyte is optionally added in excess.

Still further, it may be desirable to dilute the carrier medium by adding an additional liquid component, for instance water or one or more organic solvents, e.g. acetonitrile (ACN).

### Flow of Carrier Medium

An important feature of the present invention is that the carrier medium is under flow. The carrier medium flows along a flow path that comprises the electrode. Hence, the carrier medium as a whole can be regarded as a series of discrete, but continuous, portions of carrier fluid which successively contact the electrode during flow of the carrier medium, as a whole, into, along, and out, of the flow path.

There is no particular limitation on the nature of the flow path, beyond that it provides for the electrode to contact the carrier medium when the carrier medium flows along the flow path. The flow path can be any suitable means providing for flow of a carrier medium (e.g. a fluid carrier medium such as a liquid carrier medium).

In some embodiments, the flow path may be comprised in a device into which the electrode is integrated. For instance, such a flow path may commence at a flow inlet which is in fluid connection with a flow cell that contains the electrode, together with a counter electrode and/or reference electrode, and which is itself in fluid connection with a flow outlet. In such a device, the electrode contacts the carrier medium in the flow cell after it has flowed from the flow inlet and before it flows to the flow outlet. Continuous flow of the carrier fluid through the device provides continuous renewal of the portion of carrier fluid that is in contact with the electrode. In one particularly exemplary embodiment, the flow cell is a microfluidic cell, i.e. flow path is comprised in a microfluidic cell. One illustrative general embodiment of such a microfluidic cell is described in the present working examples.

One major advantage of the present method is that the continuous and rapidly responsive redox capacitance measurements provide for real-time sensing (e.g. concentration determination) of target particles throughout the flow period of carrier medium. Hence, as shown in the working examples, even highly transient changes in target particle concentration in portions of the carrier medium contacting the electrode can be reliably and accurately detected over an extended flow period.

The flow period as defined herein is the time over which both carrier fluid is under flow along the flow path, and thereby contacting the electrode, and redox capacitance measurements are being continuously obtained. Commonly, the flow period is at least 10 minutes, for instance at least 30 minutes or at least 60 minutes. However, there is also no particular limitation on the duration of the flow period beyond what would be useful for any particular industrial application. Hence, flow periods of at least 2 hours, at least 3 hours, at least 4 hours or at least 8 hours are also possible. There is no particular limitation in the maximum flow period, beyond what is commercially useful in a given industrial application. For instance, optionally the flow period might be no more than one week, for instance no more than 24 hours, such as no more than 12 hours.

### Electrode

The electrode functions as the working electrode in an electrochemical system, specifically a system adapted for performing redox capacitance measurements.

### Electrically Conductive Substrate

The electrode comprises an electrically conductive substrate. This substrate may comprise any electrically conducting material. The substrate may comprise a metal or carbon. The metal may be a metal in elemental form or an alloy of a metal. Optionally, the whole of the substrate comprises a metal or carbon. The substrate may comprise a transition metal. The substrate may comprise a transition metal selected from any of groups 9 to 11 of the Periodic Table. The substrate may comprise a metal selected from, but not limited to, rhenium, iridium, palladium, platinum, copper, indium, rubidium, silver and gold. The substrate may comprise a metal selected from gold, silver and platinum. The substrate may comprise a carbon-containing material, which may be selected from edge plane pyrolytic graphite, basal plane pyrolytic graphite, glassy carbon, boron doped diamond, highly ordered pyrolytic graphite, carbon powder and carbon nanotubes.

In one embodiment, the substrate comprises gold, for example the substrate is a gold substrate. However, it is also possible for the substrate to comprise other materials and so, for instance, in other embodiments, the electrically conductive substrate is not a gold substrate. Non-limiting further examples of suitable electrically conductive substrates include carbon (e.g., graphene), platinum, silver, ruthenium oxide and indium tin oxide (ITO).

The electrode surface (i.e., the substrate surface) may be planar, which includes a generally flat surface, e.g. without indentations, protrusions and pores. Such substrate surfaces can be readily prepared by techniques such as polishing with fine particles, e.g. spraying with fine particles, optionally in a sequence of steps where the size of the fine particles is decreased in each polishing step. The fine particles may, for example, comprise a carbon-based material, such as diamond, and/or may have particles with diameters of 10 μm or less, optionally 5 μm or less, optionally 3 μm or less, optionally 1 μm or less, optionally 0.5 μm or less, optionally 0.1 μm or less. Following polishing, the substrate surface may be washed, e.g. ultrasonically, optionally in a suitable liquid medium, such as water, e.g. for a period of at least 1 minute, e.g. from about 1 minute to 10 minutes. Optionally, the substrate surface may be washed with an abrasive, e.g. acidic, solution, for example following the polishing and, if used, ultrasonic washing steps. The abrasive solution may comprise an inorganic acid, e.g. H₂SO₄, and/or a peroxide, e.g. H₂O₂, in a suitable liquid medium, e.g. water. Optionally, the substrates can be electrochemically polished, which may follow any steps involving one or more of polishing with fine particles, washing e.g. ultrasonically and/or using an abrasive solution. The electrochemical polishing may involve cycling between an upper and lower potential until a stable reduction peak is reached, e.g. an upper potential of 0.5 V or more, optionally 1 V or more, optionally 1.25 V or more, and a lower potential of 0.5 V or less, optionally 0.25 V or less, optionally 0.1 V or less.

### Redox-Active Receptors

The electrode comprises redox-active receptors that are capable of capable of binding to the target particles to change the redox capacitance of the electrode.

As those skilled in the art would be aware, a wide range of suitable such redox-active receptors have already been developed and utilised in the art in connection, for instance, with voltammetric techniques and/or methods performed in under non-flow conditions (carrier medium is not under flow). Such redox-active receptors can be used in the present invention without limitation, whereupon they are also amenable to redox capacitance measurements rather than voltammetric measurements and to methods performed under flow conditions. It is not therefore necessary to provide a comprehensive survey of all such well known redox-active receptors that are capable of binding to target particles.

Briefly, nonetheless, such redox-active receptors typically have the following characteristics:
(a) They comprise a redox-active portion (sometimes known as a redox transducer), which gives rise to a characteristic redox capacitance when the redox-active receptor is comprised on the electrode and its redox capacitance probed.
(b) They further comprise an target particle binding moiety, optionally linked to the redox-active portion via a linker moiety, the target particle binding moiety being capable of binding to target particles and, when so-bound, changing the electrochemical properties of the redox-active portion (e.g. its redox capacitance characteristics).
(c) They are capable of stably associating with the electrode substrate, e.g. via an electrode substrate binding moiety (for instance, being capable of forming a self-assembled monolayer, SAM, on the electrode substrate).

A comprehensive review of redox-active receptors that are capable of capable of binding to the target particles (e.g., ions) has recently been published by Hein et al. in Chem Rev. 2020 Feb. 12; 120(3):1888-1935. This publication is herein incorporated by reference in its entirety. Furthermore, all of the redox-active receptors described in this document are herein incorporated specifically by reference as suitable redox-active receptors for use in the method of the present invention.

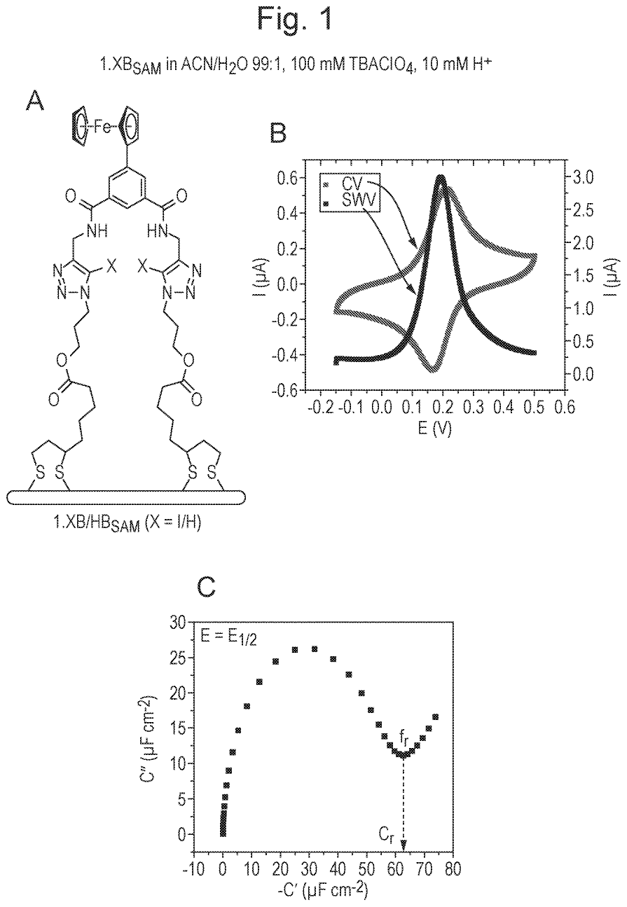

Non-limiting examples of suitable redox-active receptors are those comprising a redox active portion that is selected from: a ferrocene group, a group derived from methylene blue; a quinone; and a metallic chemical complex comprising a transition metal, wherein the transition metal is preferably Fe, Ru, Ti, V, Mn, Cr, Co, Ni, Nb, Mo or Os. One class of redox-active receptors of particular interest are those comprising a ferrocene group, for instance ferrocene-isophthalamide-triazole self-assembled monolayers (SAMs) or ferrocene-isophthalamide-iodotriazole self-assembled monolayers (SAMs), such as those having the chemical formula shown FIG. 1A (where X═H corresponds to a ferrocene-isophthalamide-triazole self-assembled monolayer and where X═I corresponds to a ferrocene-isophthalamide-iodotriazole self-assembled monolayer).

Further examples of suitable redox-active receptors include those comprising tetrathiafulvalene, porphyrins (free base or metallo-porphyins), complexed metals (particularly those of the first and second row transition metals, for instance complexed to nitrogenous ligands such as bipyridine or terpyridine) and organic redox-active moieties (including tri (alkyl/aryl) amines and diquat/paraquat).

As will be evident to those skilled in the art, the electrode discussed in detail herein is a working electrode and the method of the invention involves obtaining redox capacitance measurements of the electrode in the further presence of a reference electrode and/or a counter electrode.

### Redox Capacitance Measurements

In the method of the invention, step (C) comprises continuously obtaining redox capacitance measurements of the electrode over the flow period, thereby sensing the binding of target particles to the redox-active receptors and hence the presence, or absence, of target particles in the portion of carrier medium that is in contact with the electrode at each time point over the flow period. As discussed elsewhere herein, and empirically demonstrated in the examples section, application of redox capacitance as the property being probed electrochemically has been found to lead to significant advantages in the context of sensing particles under continuous flow conditions.

The electrochemical response of the system is sensitive to the presence of the target particles both in the carrier medium as a whole and in different portions of the carrier medium as they flow along the electrode and successively contact the electrode. More specifically, the redox capacitance properties of the electrode are altered (e.g. being cathodically displaced) if target particles are present and therefore bind to the receptors of the electrode. Thus, if (a portion of) the carrier medium does contain the target particles then a particular experimental measurement will be obtained (when that portion is contact with the electrode). On the other hand the measurement will be different if the (portion of the) carrier medium does not contain the target particles (when that portion is contact with the electrode).

Similarly, changes in the measurement will occur as the concentration of the target particles in the carrier medium changes. Conveniently, the changes as a function of target particle concentration can be quantified by way of a series of control experiments performed using carrier medium containing known concentrations of target particles, which enables preparation of a calibration curve showing the results as a function of concentration and which can therefore be applied to establish the concentration of target particles in a test carrier medium from the electrochemical measurements made on that system. The working examples section empirically demonstrate how the modulations in redox capacitance can readily be correlated to concentration of target particles. Hence, in a preferred embodiment of the invention, the method step (C) comprises determining the concentration of target particles in the portion of the carrier medium that is in contact with the electrode at each time point over the flow period.

Redox capacitance refers at its most general to an electrochemical method which comprises interrogating (by electrochemical means) the redox capacitance, Cᵣ, and/or related measurement parameters such as C′ and/or C″, of the electrode defined herein. The electrode possesses an intrinsic redox capacitance as a result of the presence of the redox-active receptors. The redox capacitance of the electrode is modified if target particles bind to the receptor.

Redox capacitance is known in the art and the present document does not seek to be a primer on its scope or specific application. In general, the method of the invention encompasses any method that involves interrogating the redox capacitance of the electrode in order to determine whether the electrode comprises bound target particles. Representative examples of disclosure relating to application of redox capacitance spectroscopy for detecting target particles (but though not describing application of such techniques to particle sensing under flow, still less foreshadowing the significant resulting advantages of so-doing) can be found in WO 2015/022483, WO 2016/120606, J. Phys. Chem. B 2012, 116, 30, 8822-8829, Biosensors and Bioelectronics 50 (2013) 437-440, Biosensors and Bioelectronics 57 (2014) 96-102, WO 2019/145706 and WO 2021/152320, the contents of all of which are herein incorporated by reference in their entirety. Further guidance on exemplary, but non-limiting, specific redox capacitance principles can found in the working examples of this disclosure.

In one typical embodiment of the present invention, the continuous redox capacitance measurements are obtained with a time scan at fixed potential (e.g., the half-wave potential, E_{1/2}, or at a potential displaced therefrom as discussed further below and in the working examples) and fixed frequency (e.g. fᵣ, the frequency at the redox capacitance, Cᵣ, e.g. as determined by electrochemical impedance spectroscopy). In a preferred embodiment, the continuous redox capacitance measurements are corrected for baseline degradation, such as by linear scaling of all data points to the initial baseline immediately prior to commencement of the flow period.

The working examples provide a detailed demonstration of how, for various exemplified combinations of particular target particles and particular electrodes (functionalised with particular redox-active receptors), continuous redox capacitance measurements can be obtained over a flow period in which the carrier medium is flowing along the flow path, and how those measurements can be correlated to sensing of the target particles, including determining their concentration in the carrier medium and establishing the limit of detection (LOD) for particular such systems. Those skilled in the art would immediately appreciate that these principles can be readily adapted to perform analogous methods for alternative combinations of target particles and electrodes (including those functionalised with different redox-active receptors). Hence, the methods exemplified in the working examples are clearly generalizable to detection of target particles by continuous redox capacitance measurement methods.

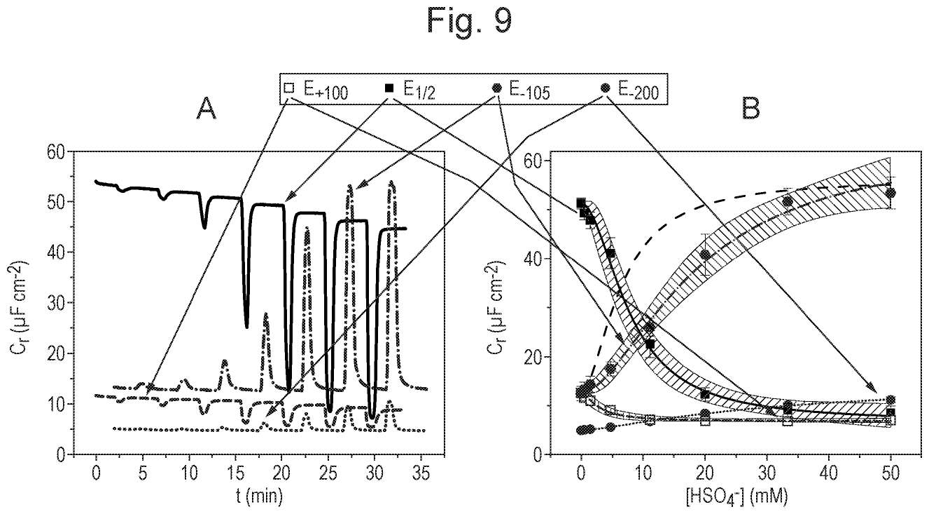

Furthermore, the working examples also demonstrate that aspects of the continuous redox capacitance measurements can be adjusted in order to optimise the method of the invention to a particular embodiment. For instance, aspects of the continuous redox capacitance measurements that can be adapted include the choice fixed potential at which the measurements are obtained (see especially example sections S7 to S9, Tables 1 and S6.5 and FIGS. 9, 12 and 13), the choice of AC frequency, and the choice of AC amplitude. As shown in the examples, routine optimisation of these features of the redox capacitance measurement can be used for any specific embodiment of the invention (for instance, where particular target particles are of interest, and perhaps where other non-target particles may also be present in the carrier medium) in order to maximise the sensitivity and selectivity of the method to the desired application. For instance, such optimisation may result in optimisation of the limit of detection, the maximum duration of the flow period over which the measurements can be performed without confounding signal degradation, and/or the selectivity of the sensing targets the target particles in preference to other particles that may be present in the carrier medium.

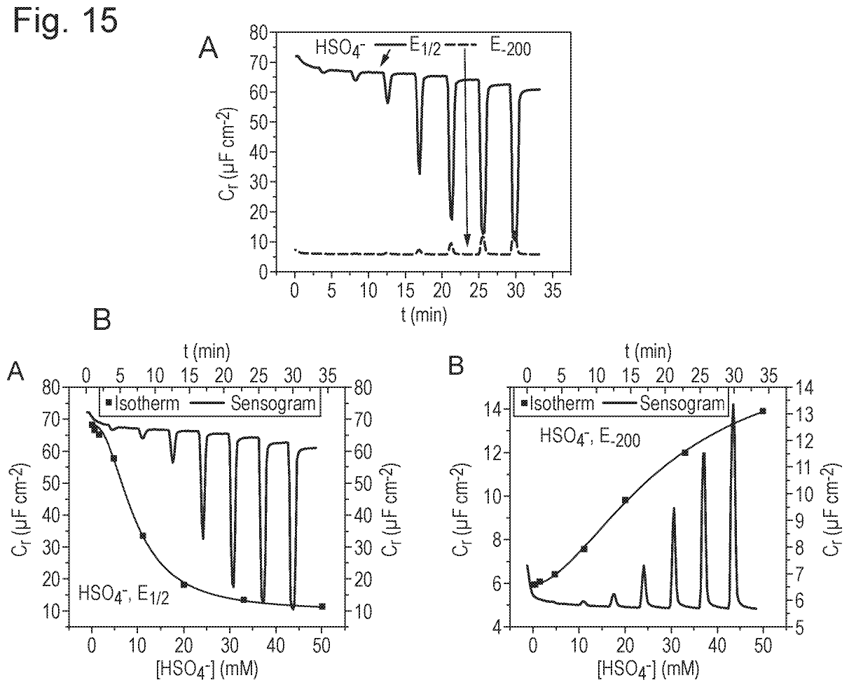

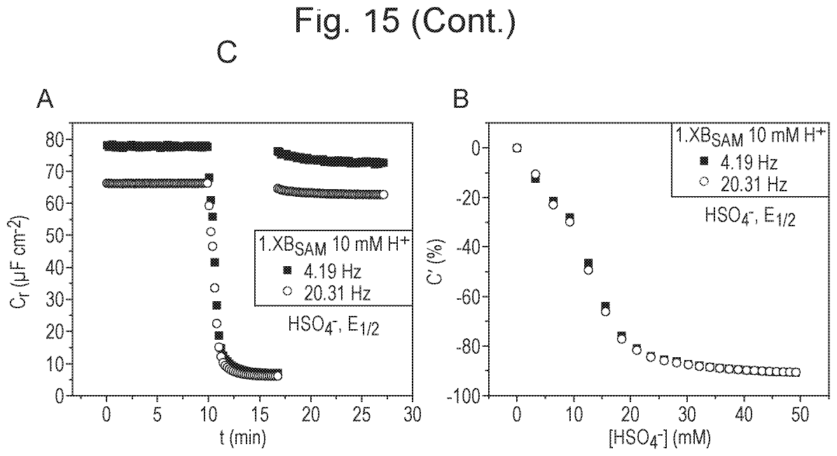

Still further, the continuous redox capacitance measurements may comprise obtaining measurements not only at a single combination of (single) electrode potential, (single) AC frequency and (single) AC amplitude, but may involve more complex measurements being obtained, involving cycling between two or more electrode potentials, AC frequencies and/or AC amplitudes. Section S10 of the working examples exemplifies two such possibilities, in particular the cycling between two or more electrode potentials (see also FIGS. 15A and 15B) and cycling between two or more AC frequencies (see also FIG. 15C). Such measurements may further be useful for increasing information density and/or tuning the performance of the sensing method, e.g. for sensitivity and/or selectivity. Accordingly, the method of the invention may comprise obtaining redox capacitance measurements of the electrode in which the electrode potential is the E_{1/2} potential of the electrode. Alternatively (when measurements are performed at a single potential), or additionally (when measurements are performed at multiple potentials), the method may comprise comprising obtaining redox capacitance measurements of the electrode in which the electrode potential is different from the E_{1/2} potential of the electrode. For instance, the electrode potential different from the E_{1/2} potential of the electrode may be potential that lies between: (a) the E_{1/2} potential of the electrode; and (b) the E_{1/2} potential of the electrode in the presence of an excess of target particles (i.e. under conditions where the shift in the E_{1/2} potential caused by binding of target particles is strongly or maximally exhibited).

The redox capacitance measurements of the electrode may be obtained at a single electrode potential. Alternatively, the redox capacitance measurements of the electrode may be obtained at two or more electrode potentials by cycling between the two or more electrode potentials over the flow period.

Furthermore, the redox capacitance measurements of the electrode may be obtained at a single (AC) frequency. Alternatively, the redox capacitance measurements of the electrode may be obtained at two or more frequencies by cycling between the two or more frequencies over the flow period.

Still further, the redox capacitance measurements of the electrode may be obtained at a single AC amplitude. Alternatively, the redox capacitance measurements of the electrode may be obtained at two or more AC amplitudes by cycling between the two or more amplitudes over the flow period.

Still further any combination of the above single or two or more potentials, single or two or more AC frequencies, and single or two or more AC amplitudes can be adopted without limitation.

When carrying out the method of the invention it has been found that advantageous limits of detection (“LOD”) can be achieved. For instance, in preferred embodiments the limit of detection of the target particles may be lower than 100 μg/mL, preferably lower than 75 μg/mL and more preferably still lower than 50 μg/mL (e.g. approximately 30 μg/mL or lower). For a particular embodiment (target particle identity, working electrode construction, etc.) LOD can be determined, for instance, according to LOD=3σ/s, where s is the slope of the linear region of the calibration curve for concentration of the target particles as a function of redox capacitance measured and σ the standard deviation of the blank.

### EXAMPLES

Ubiquitous in nature, anions play vital roles in many natural and technological processes, necessitating their sensitive and selective detection across applications relevant to the environment, healthcare, and many others.

Voltammetric sensing using redox-active receptors has previously been explored as a possible solution for detection of anions under flow. A specific advantage of this approach is the modulation of the receptor's ion binding properties upon an in situ switching of the redox state. The general utility of this approach is now well established for the sensing of a range of anions both in solution and at interfaces. Importantly, the surface immobilisation of redox-active ion receptors is not only associated with significant signal enhancement effects, but also presents a more useful platform for real-life device integration. Nevertheless, continuous ion sensing via voltammetric techniques is challenging. Specifically, as transient, non-equilibrium techniques, cyclic voltammetry or pulsed voltammetric methods such as differential pulse (DPV) or square-wave voltammetry (SWV), cannot generally be utilised in a simple, continuous signal generating fashion, but instead re-quire significant data analysis/processing.

Herein we demonstrate, in a proof-of-principle, that the use of a redox-capacitive methodology can be used for the sensing of ions. This is associated with the combined advantages of both voltammetric and impedimetric techniques and enables not only a direct tuning of the ion binding (and sensing performance) via specific surface polarisation, but also real-time, continuous-flow ion sensing with high sensitivity and a simple, direct sensor readout.

### Results and Discussions

In a recent study we reported on the utility of redox-active halogen bonding (XB) and hydrogen bonding (HB) ferrocene-isophthalamide-(iodo) triazole self-assembled monolayers (SAMs) 1.XB/HB_{SAM} as potent voltammetric anion sensors (FIG. 1A) (see Hein et al. Chem. Sci. 2021, 12, 2433-2440 and Patrick et al. ChemRxiv 2021, DOI: 10.33774/chemrxiv-2021-2nqwc). In the aqueous-organic solvent mixture ACN/H₂O 99:1, well-defined, and large scale, cathodic perturbations of the Fc/Fc⁺ redox couple (FIG. 1B) in the presence of various oxoanions and halides were observed. We demonstrated that the addition of small amounts of acid (100 μM-10 mM HClO₄) significantly enhanced the voltammetric stability of the films, with minimal effect on the sensor response towards anions of low basicity, such as HSO₄⁻.

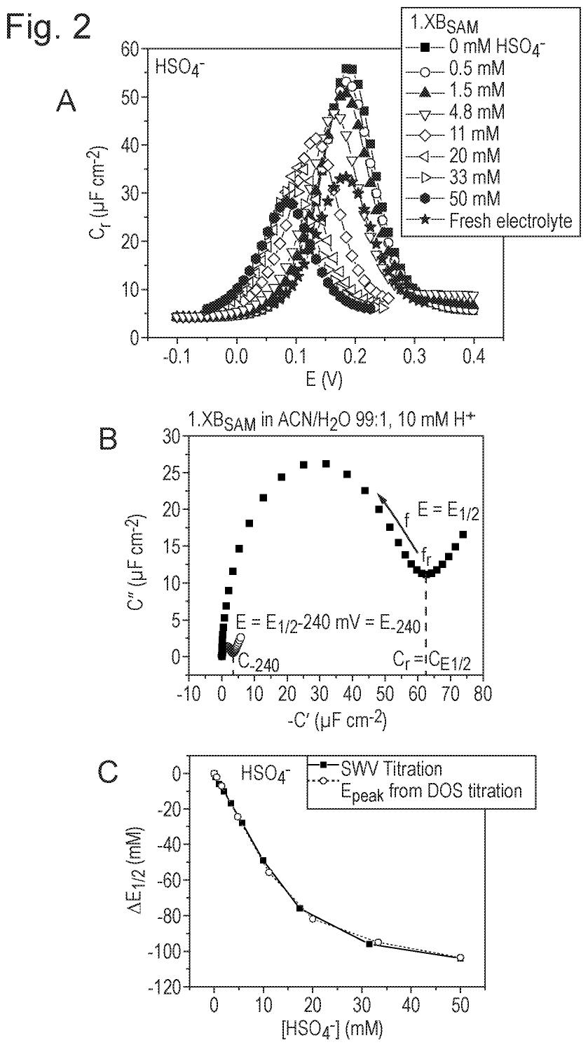

As depicted in FIG. 1C, impedance-derived capacitance spectroscopy can resolve the (redox) capacitive fingerprints of the electroactive SAM. Specifically, this technique operates at a fixed electrode polarisation (DC potential) upon which a small amplitude (10 mV) AC perturbation of a specific frequency is superimposed. By scanning different frequencies across a large range (here 100 kHz-1 Hz) capacitive Nyquist plots can be obtained (FIG. 1C), wherein the diameter of the semi-circular region corresponds to the interfacial (redox) capacitance, a parameter reporting directly on the Faradaic charging capacity of the film (see: (a) Bueno et al. Anal. Chem. 2014, 86, 1337-1341; (b) Lchr et al. Anal. Chem. 2014, 86, 2559-2564; (c) Bueno et al. Anal. Chem. 2018, 90, 7095-7106; (d) Bueno et al. The Nanoscale Electrochemistry of Molecular Contacts. Springer: 2018). This redox capacitance is largest when the ratio of oxidised and reduced receptor states is equal, i.e. at the half-wave potential (E_{1/2}), where a Cᵣ of 62.6 μF cm⁻² is obtained (FIG. 1C), approximately 20-fold larger than the non-Faradaic film capacitance (obtained if the electrode is polarised in a regime devoid of any Faradaic activity, herein 3.4 μF cm⁻², see FIG. 2A).

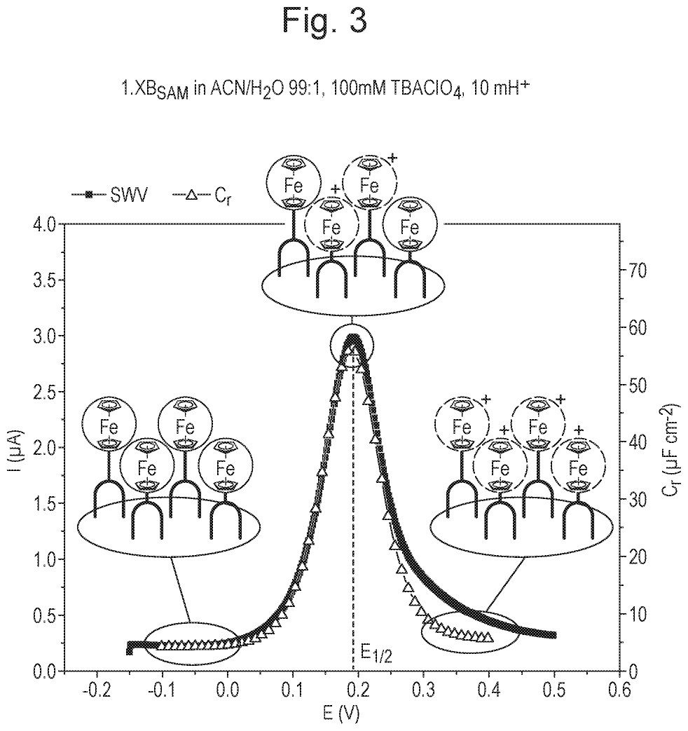

The redox density of states (DOS), the variance of Cᵣ with the electrode potential, can be resolved by a single frequency capacitance measurement (at fᵣ) at different potentials (instead of recording a whole Nyquist plot), as shown in FIG. 3 (triangles). This redox capacitance distribution tracks, as expected, the same Gaussian distribution as a standard SWV measurement and reports on the relative proportion of oxidized and reduced receptor states.

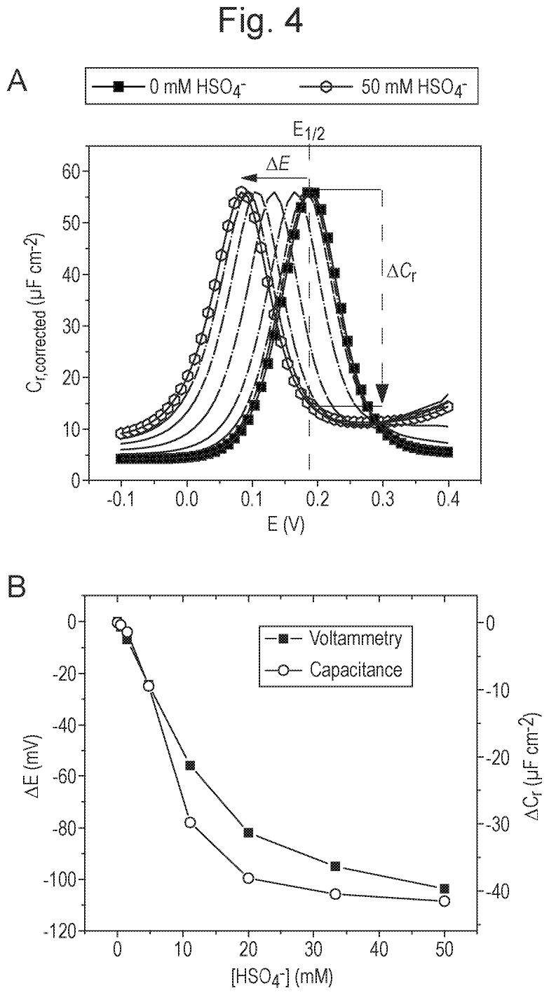

Upon anion binding (herein exemplary HSO₄⁻ at 1.XB_{SAM}), this redox capacitance distribution shifts cathodically, as shown in FIG. 4A, whereby the magnitude of the potential shift is identical to that obtained by standard voltammetric analysis (FIG. 4B, trace with square data points). Thus, in the first instance, anion sensing experiments carried out in this manner afford information analogous to that accessible through SWV (or similar voltammetric analyses), i.e. both report on the anion binding induced cathodic potential shift of the Fc/Fc⁺ redox transducer (FIG. 2C).

However, in contrast to the transient nature of voltammetry, the redox-capacitive approach presented herein does not require a “sweeping” of the voltage and can be conducted at any freely-chosen and fixed electrode potential (at a static equilibrium). Specifically, Cᵣ can be continually recorded at a fixed electrode polarisation (and frequency) that is, at any specific point within the Gaussian redox distribution shown in FIG. 3. Importantly, this signal is also highly responsive to anion binding. For example, when the electrode is polarized at E_{1/2}, anion binding induces a continuous, monotonic decrease in Cᵣ (C_{E1/2}; FIG. 4A, arrow, and FIG. 4B, star trace), as the DOS distribution is continually shifted towards cathodic potentials, away from E_{1/2}, such that C_{E1/2} now lies within the anodic tail of the Gaussian distribution.

As expected, the binding/sensing isotherm obtained in this manner is similar to that obtained by voltammetric analysis (FIG. 4B, square trace), though with a somewhat steeper and more quickly plateauing response, arising from an amplification effect, as discussed in more detail in a later section.

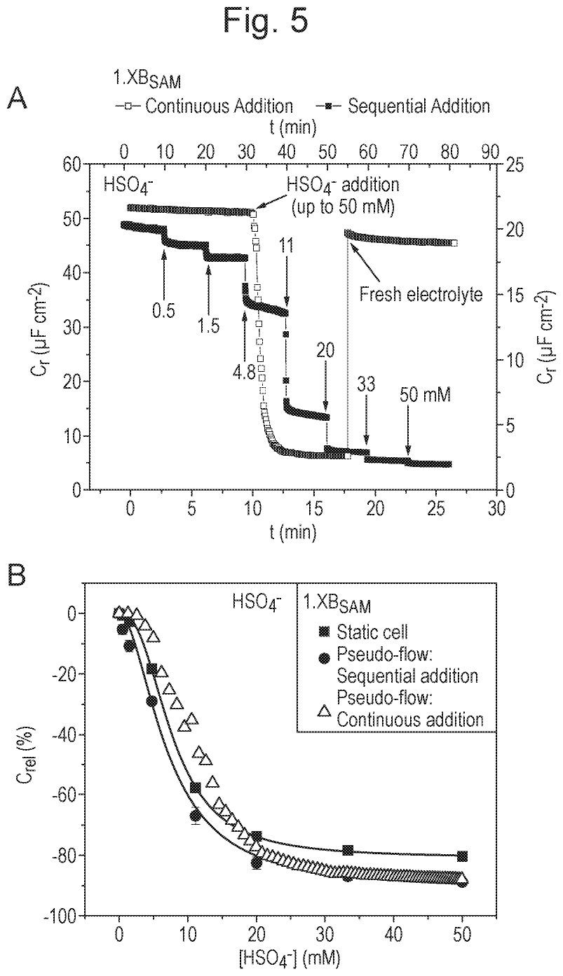

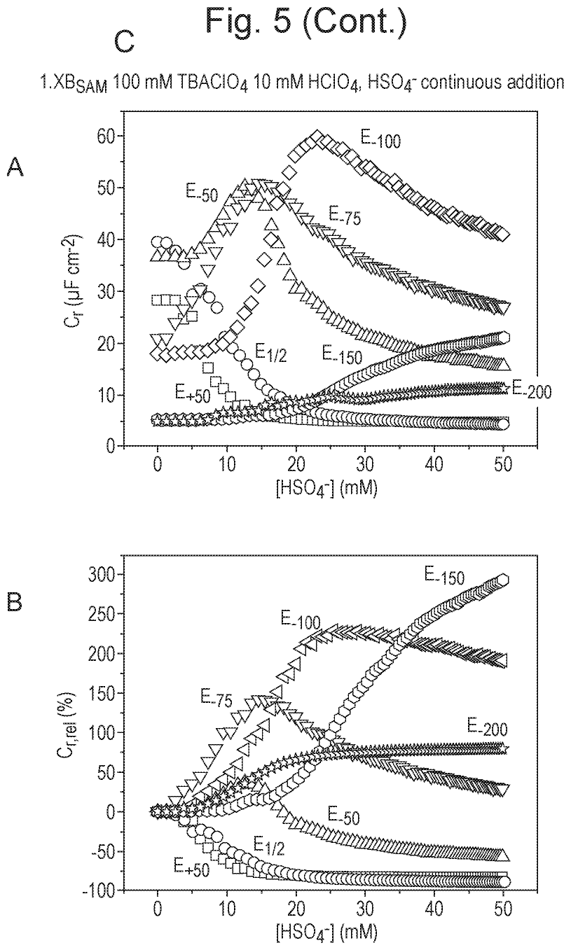

Importantly, this single frequency, fixed-potential redox capacitance methodology not only provides a simple, direct, and non-perturbatory sensor readout (Cᵣ), proportional to the analyte concentration, but is also associated with a high temporal resolution (typically ≤2 s, faster than that of highly optimised voltammetric methods—see Patrick et al. ChemRxiv 2021, DOI: 10.33774/chemrxiv-2021-2nqwc). This presents a particularly useful, fast and experimentally straightforward way of obtaining detailed information about interfacial binding events, an analysis that cannot be easily carried out by standard voltammetric methodologies. Specifically, in a stirred electrochemical cell Cᵣ can be monitored upon continuous analyte addition with the help of a syringe pump, affording sensing isotherms with a very high number of data points (see FIGS. 5A-C and Section S4 for associated discussions). Furthermore, the fast, direct signal readout enables both a continuous, real-time sensing under flow and an ability to tune binding affinities and sensor performance, as demonstrated below.

### Continuous, Real-Time Anion Sensing Under Flow

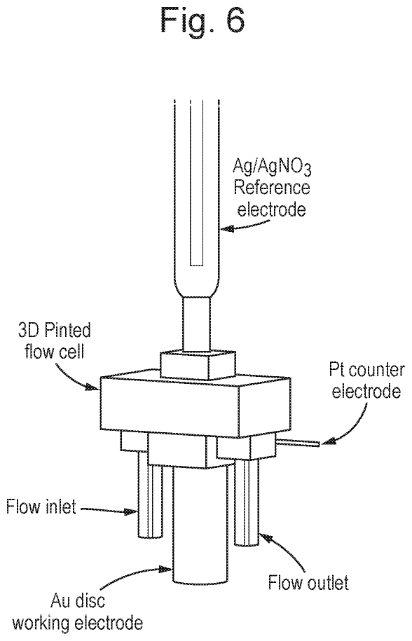

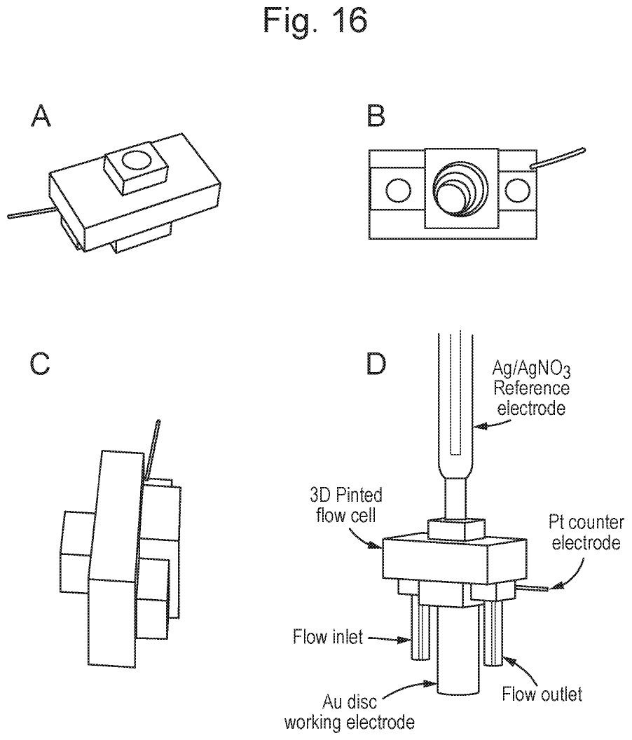

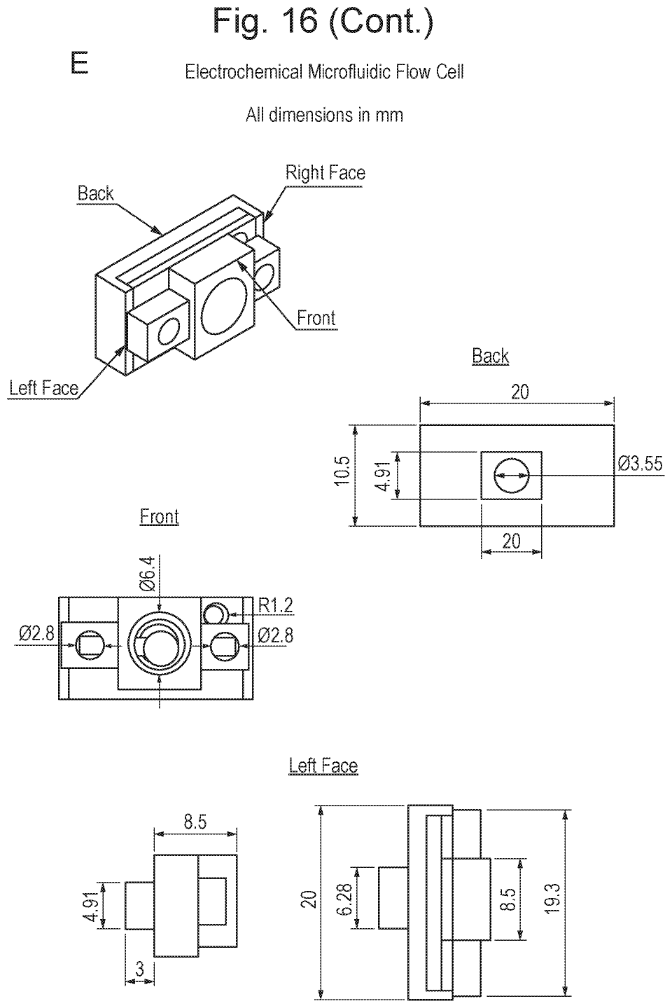

To this end, we have designed and 3D-printed a custom electrochemical flow cell with an internal chamber of 100 μL (FIG. 6), through which electrolyte (ACN/H₂O 99:1, 100 mM TBAClO₄, 10 mM HClO₄) can be continuously pumped (for further information see FIG. 16, FIG. 9 and Sections S1.7 and S1.8).

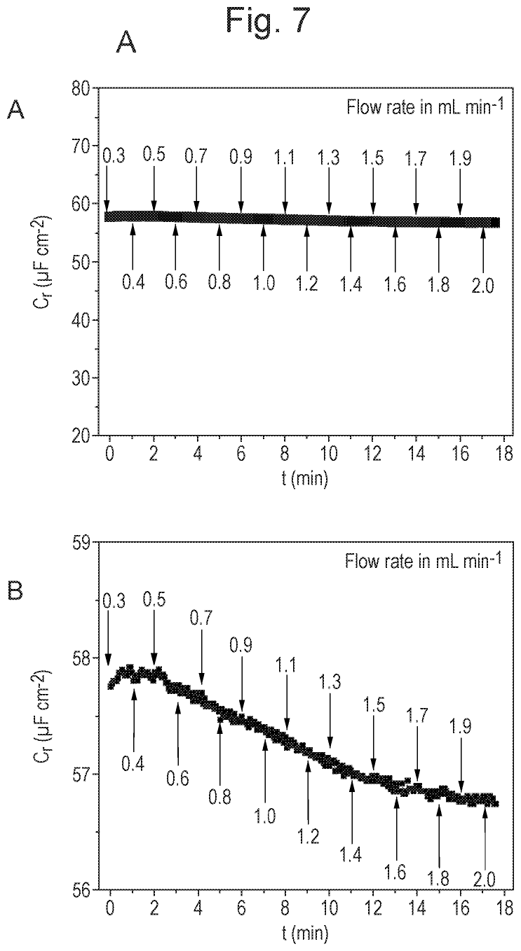

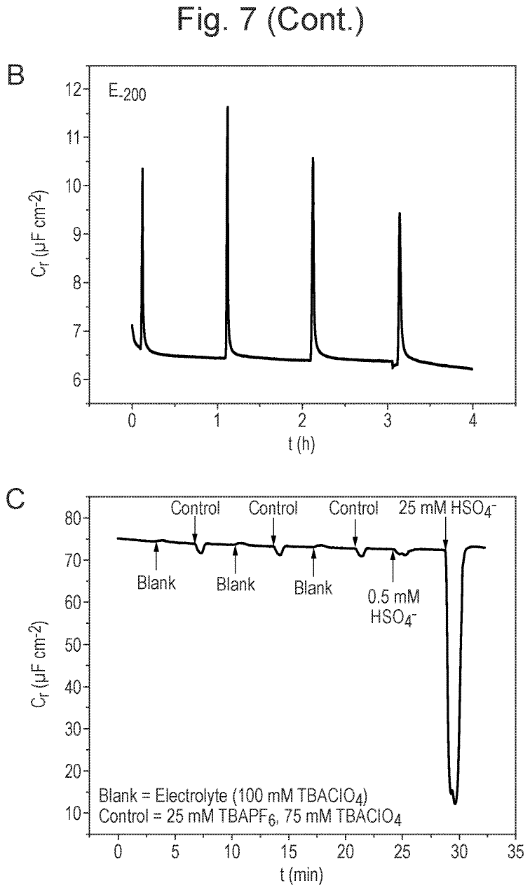

These analyses were tolerant to a large range of flow rates (up to 2 mL min⁻¹) and displayed a stable (flow rate-independent) capacitance response (measured at fixed-frequency (fᵣ) and fixed-potential: see FIG. 7A). With the help of a sample injection valve, aliquots of analyte solution of defined volume (typically 500 μL) were injected.

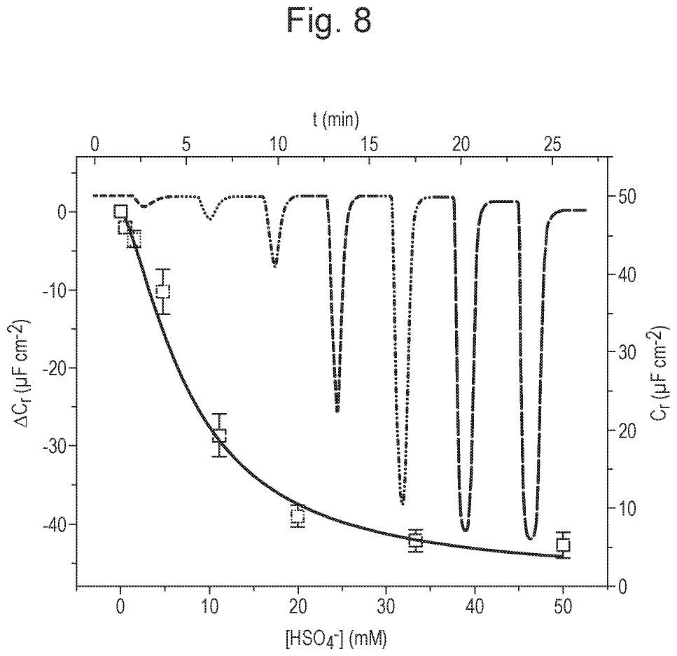

When these measurements were carried out at a constant electrode polarisation at E_{1/2}, the exposure to analyte (HSO₄⁻) was associated with notable response spikes (signal decrease), as shown in FIG. 8, while, as expected, control injections of the non-coordinating PF₆⁻ did not induce a significant capacitance response (FIG. 7B). The injection of the analyte sample is immediately followed by a “washing” step with fresh electrolyte, such that a (new) Cᵣ baseline is (re) established. The signal degradation overtime is in general sufficiently minimal so as to support analyses over hours (see FIG. 7C).

As alluded to above, a differing sensing performance of the redox capacitance readout is also apparent in a comparison of the shape of the Cᵣ response isotherms to the standard voltammetric response isotherms (FIG. 4B). The former notably plateaus quicker, indicative of stronger anion binding (vide infra). The limit of detection (LOD) of this redox capacitive flow sensor towards HSO₄⁻ is 44.5±15.5 M (see Sections S2 and S6 for details), lower than that attainable by even highly optimized, laborious voltammetric methods (Table 1).

TABLE 1
Apparent binding constants Kₐₚₚ (M⁻¹) and maximum relative response
ΔCₘₐₓ (μF cm⁻²) of 1.XB_{SAM} towards HSO₄⁻ obtained
by fitting of the response isotherms (FIG. 9) at different potentials
according to the Langmuir-Freundlich model (Eqn. 1).

| Redox capacitance | | | | | Voltammetry |
|---|---|---|---|---|---|
| E_{dc} | E_{± 100} | E_{1/2} | E₋₁₀₅ | E₋₂₀₀ | n.a. |
| LOD (μM) | 42.4 ± 4.0 | 44.5 ± 15.5 | 70.4 ± 18.2 | 78.9 ± 29.1 | 59.8 ± 5.5^{a} |
| Kapp (M− 1) | 320 ± 60 | 121 ± 6 | 64.2 ± 7.7 | 36.2 ± 7.2 | 68.6 ± 17.6 |
| ΔCₘₐₓ (μF | − 6.09 ± 0.35 | − 44.4 ± 1.0 | 46.0 ± 3.05 | 8.95 ± 1.12 | −116 ± 9^{c} |
| cm^{- 2})^{b} | | | | | |

The limit of detection (LOD) was obtained by analysis of the pseudo-linear region at low concentration (0-4.8 mM, see Section S2.3).
The sensor performance in an optimised voltammetric format (SWV) is also shown for comparison (Patrick et al. ChemRxiv 2021, DOI: 10.33774/chemrxiv-2021-2nqwc).
N.a.—not applicable.
Errors represent one standard deviation of at least three independent repeats. Further details are provided in Section S6.
^{a}under highly optimised conditions with advanced data processing/fitting.
Without this optimised analysis the LOD is significantly worse (825 ± 138 μM - Patrick et al. ChemRxiv 2021, DOI: 10.33774/chemrxiv-2021-2nqwc).
^{b}at [HSO₄⁻] −> ∞ obtained from fitting.
^{c}in mV.

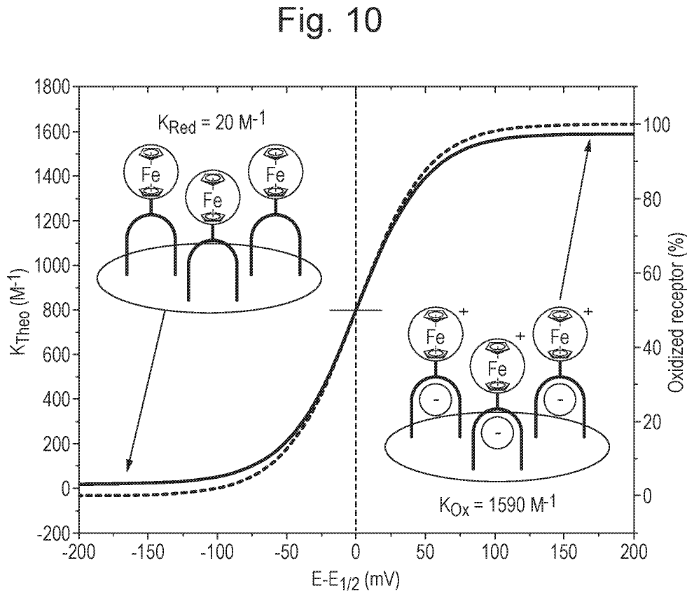

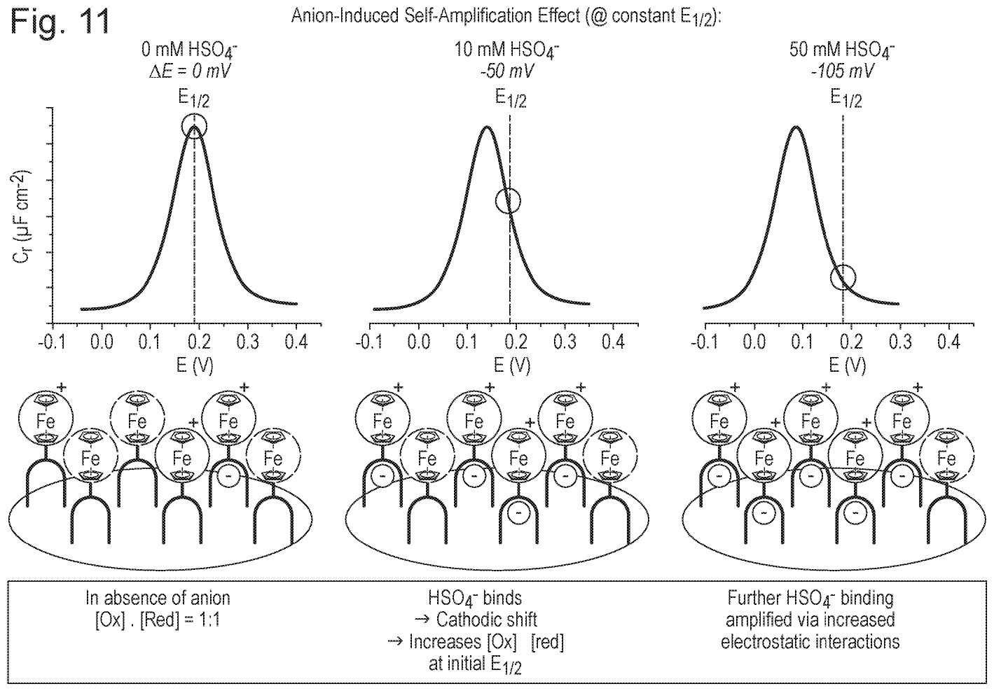

Anion binding to the electroactive interfaces herein is principally governed by two separate binding equilibria, namely anion binding to the oxidised receptor 1.XB_{SAM}⁺ (K_{Ox}, here ≈1,580 M⁻¹) as well as to the native, neutral receptor 1.XB_{SAM} (K_{Red}, here ≈20 M⁻¹) (see Hein et al. Chem. Sci. 2021, 12, 2433-2440). The overall, apparent anion binding strength Kₐₚₚ is then dependent on the ratio of the two receptor oxidation states [Ox]/[Red] (i.e. 1.XB_{SAM}⁺/1.XB_{SAM}), in turn determined by the Nernst equation, being unity at E_{1/2} (FIG. 10). Upon anion binding the formal potential at which this ratio remains unity shifts cathodically, such that the initial (and constant) electrode polarisation of E_{1/2} now lies in the anodic tail of the Nernstian redox distribution (FIG. 11).

Thus, the interface is, as a consequence of anion binding, rendered more cationic at the constantly imposed potential (as the ratio of 1.XB_{SAM}⁺/1.XB_{SAM} increases) and thus displays a higher overall anion binding affinity. In effect, anion binding is, at least phenomenologically “self-amplified”. Standard voltammetric analysis, where the apparent binding remains constant, offers no such effects (see Section S7 for further discussions).

It is crucial to note that this potential-dependent binding modulation does not arise from any specific interaction of the anion with the electrode itself (i.e. the individual K_{Ox} and K_{Red} are unaffected), but is purely governed by the potential-dependence of the ratio of the receptor oxidation states.

As discussed above, these anion binding induced progressive changes in binding affinity are qualitatively reflected in a steeper sensor response isotherm and can be quantified by fitting of the binding isotherms according to the Langmuir-Freundlich model (Eqn. 1), which in all cases afforded good fits (Table 1 and Tables S6.1-S6.4). From this analysis the apparent anion binding constant Kapp to the receptive interface was obtained, which as shown in Table 1, is for Cᵣ measurements carried out at E_{1/2}, significantly larger (121±6 M⁻¹) than that of the standard voltammetric format (68.6±17.6 M⁻¹).

$$θ = \frac{{( {K}_{app} * [ {A}^{-} ] )}^{n}}{1 + {( {K}_{app} * [ {A}^{-} ] )}^{n}} \qquad Eqn . 1$$

This confirms an overall binding (and response) enhancement, arising from the “self-amplification” effect in the redox capacitive method, which, together with a natively higher sensitivity of the methodology is responsible for enhanced responses and lowered LODs (Table 1 and Tables S6.1-S6.5). Importantly, the dependence of Kₐₚₚ on the ratio of 1.XB_{SAM}⁺/1.XB_{SAM} can be further exploited to fine-tune anion binding strength and sensor performance by judicious choice of the electrode potential.

### Tuning Sensor Performance Through Surface Polarisation

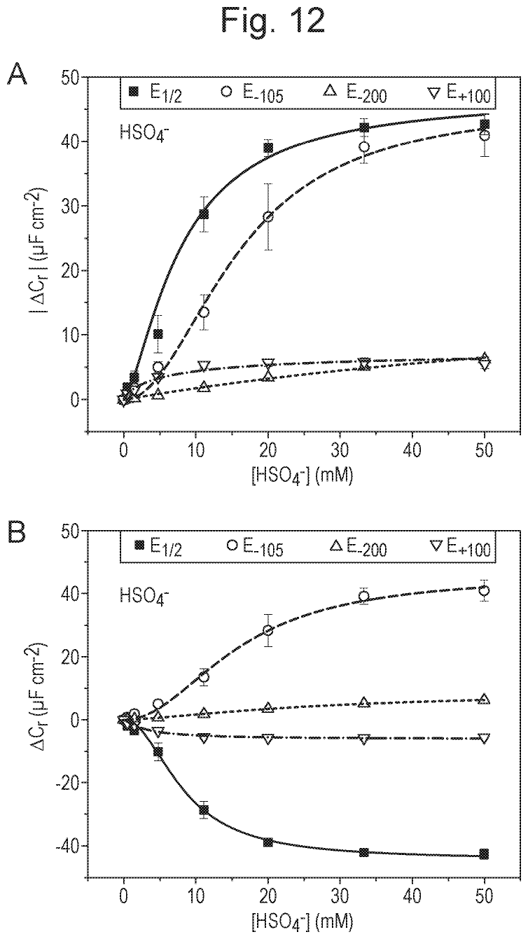

When the sensor electrodes are poised anodically with respect to E_{1/2}, e.g. at +100 mV (E+100, FIG. 9), anion binding induces a generally similar response to that at E_{1/2} (monotonic signal decrease). However, with a smaller overall response magnitude (ΔCₘₐₓ), as the baseline Cᵣ signal at this potential is already smaller (Table 1). However, the response isotherm is noticeably steeper, a direct reflection of the enhanced anion binding at the more Lewis acidic 1.XB_{SAM}+ (see FIGS. 11 and 12), as reflected in a Kₐₚₚ of 320±60 M⁻¹.

With the sensor poised at potentials cathodic of E_{1/2} anion binding shifts the redox distribution such that the redox capacitance grows (signal “switch-on”, FIG. 9). For example, at −105 mV (the potential to which the redox distribution shifts to upon exposure to 50 mM HSO₄⁻), a response isotherm that is in principle mirrored to that at E^{1/2}, is observed (FIG. 9, E-105 trace and dashed black line). However, as a result of the initially lower [Ox]/[Red] ratio at E-105 binding is comparably attenuated (Kₐₚₚ=64±8 M⁻¹). These effects are expectedly more pronounced at more cathodic potentials, where the initial equilibrium is shifted further towards the lower affinity [Red] redox state and increasingly higher analyte concentrations are required to induce a Cᵣ response (see FIGS. 10 and S7 for associated discussions). For example, at −200 mV a smaller response with a Kₐₚₚ of 36.2±7.2 M⁻¹ was observed (Table 1, FIG. 9, E-200 trace), corresponding to an almost ten-fold modulation of HSO₄⁻ binding affinity (cf. Kₐₚₚ, E+100=320 M⁻¹).

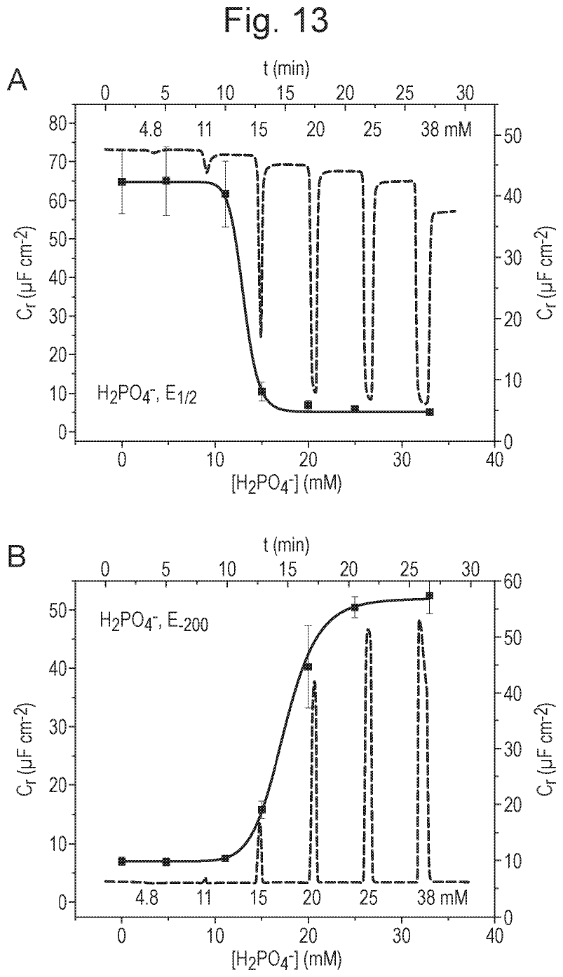

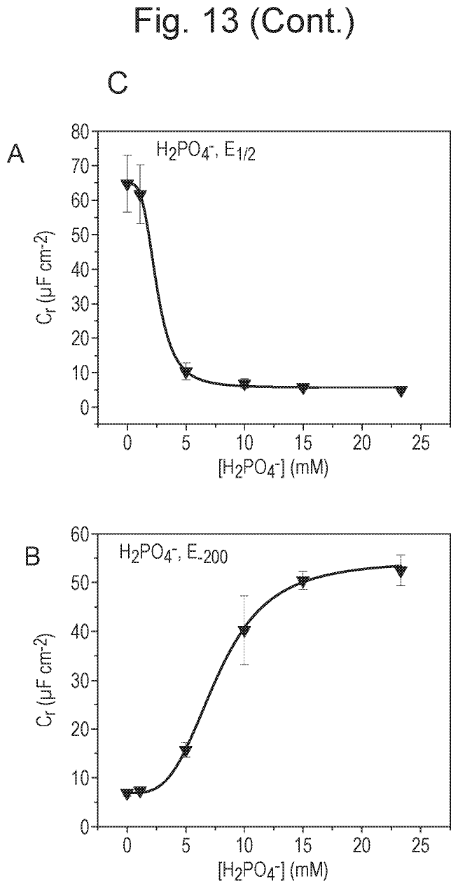

This unique control over guest binding strength is, of course, extendable to other anions. For example, binding of H₂PO₄⁻ to the interface is similarly tunable by variation of the electrode potential, with significantly stronger Kₐₚₚ=401±35 M⁻¹ at E_{1/2} than at a cathodic polarisation (132±29 M⁻¹ at E-200), see FIG. 13. We also envision that this potential dependence of binding strength can be exploited to tune sensor selectivity.

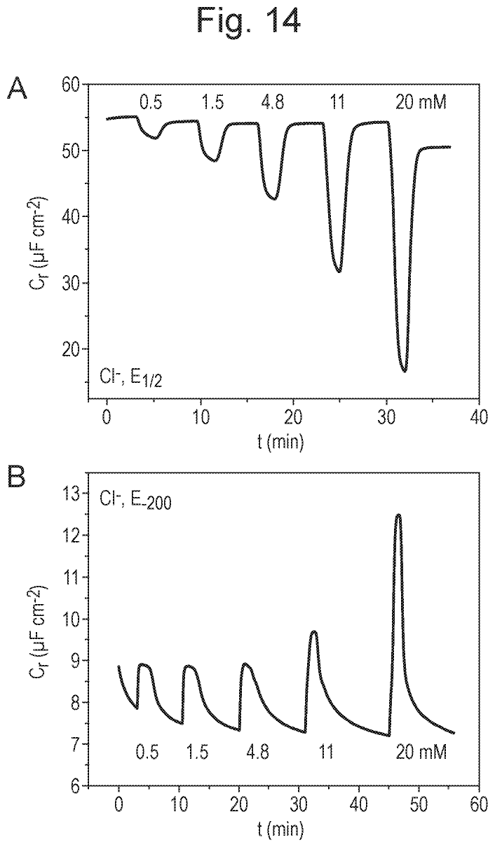

In addition to supporting an unprecedented ability to monitor selective anion binding under flow and with tuneable affinity, the methods outlined offer a temporal resolution sufficient to resolve interfacial guest association/dissociation kinetics, something that remains particularly challenging. For example, as depicted in FIG. 14, the sensor response towards Cl is slightly different to that of the oxoanions and is characterised by a slower baseline recovery upon washing with fresh electrolyte, an observation we tentatively assign to slow anion (de) complexation (see Section S9.2 for further discussions).

The methodology introduced here for anion sensing at a halogen bonding interface is equally translatable to a wide variety of other redox active interfaces, solvent systems and analytes, wherein the voltammetric profile is reversibly perturbed by guest binding. Significantly, as demonstrated, assaying is markedly enhanced and more facile than that accessible through standard voltammetric methods, is supportive of a continuous sensor output, and enables binding thermodynamics to be both mapped and tuned. The latter can be further expanded by choice of different AC frequencies or amplitudes as well as simultaneous measurements at multiple frequencies or potentials offering an even higher information density and additional tuneability (see for example FIG. 15, and Section S10 for associated discussions).

### CONCLUSIONS

We have demonstrated herein that redox capacitive sensing is a highly adaptable, novel approach for the detection of ions. It is notably associated with a number of unique advantages over standard electrochemical methodologies, including a high sensitivity and a facile and continuous signal read-out. Ion induced shifts in the film redox density of states energy distribution can be monitored from any initially applied, constant electrode polarisation. This not only facilitates assays which are either “signal switch-on” or “signal switch-off”, but also supports both an ability to tune the thermodynamics of ion recruitment and a “self-amplification” in how this is reported by the sensor. Specifically, a judicious choice of the electrode polarisation facilitates a direct tuning of anion binding affinities over one order of magnitude, thereby modulating sensor performance and analytical sensitivity.

Exemplified herein with the continuous flow sensing of bisulfate at a redox active halogen-bonding ferrocenyl anion receptive film, this approach importantly is applicable to ion assaying in general. We believe this redox capacitance methodology has significant transformative potential for a wide range of sensing applications, as well as being a potent new analytical tool for supramolecular host-guest ion binding.

### Supporting Information Sections S1 to S11

### S1—EXPERIMENTAL

### S1.1: General Information

All experiments were performed at room temperature in the presence of oxygen. All commercially available chemicals and solvents were used as received without further purification. All hygroscopic tetrabutylammonium (TBA) salts were stored in vacuum desiccators at room temperature. Ultrapure water was obtained from a Milli-Q system (18.2 M (2 cm). Supporting electrolyte (TBAClO₄ from Sigma Aldrich) was of electrochemical grade. Receptors 1.XB were synthesised as described previously (see Hein et al. Chem. Sci. 2021, 12, 2433-2440).

### S1.2: Electrochemical Measurements

All experiments were conducted using an Autolab Potentiostat (Metrohm) or PalmSens4 Potentiostat with a three-electrode setup equipped with a gold disc working electrode (BaSi, 1.6 mm diameter) and platinum wire counter electrode. A non-aqueous Ag|AgNO₃ reference electrode (with an inner filling solution of 10 mM AgNO₃, 100 mM TBAClO₄ in ACN) was utilised for all experiments. All potentials reported are wrt. to this non-aqueous Ag|AgNO₃ reference electrode. All experiments were carried out with 100 mM TBAClO₄ as a supporting electrolyte with additional 10 mM HClO₄, as indicated. In all cases, including sensing studies, the ionic strength was maintained at a constant 100 mM TBA-anion (+10 mM HClO₄) throughout.

### S1.3: Electrode Pre-Treatment and SAM Formation

Au disc electrodes were cleaned according to previously reported protocols (see Patrick et al. Chem. Eur. J. 2021, 27, 10201-10209). Immediately following the cleaning procedure, the Au disc electrodes were rinsed thoroughly with water and ethanol and immersed in a solution of 0.25 mM 1.XB in ACN overnight in the dark. Subsequently, the Au disc electrodes were rinsed with copious amounts of ACN and then used immediately. Detailed surface characterisation of the so-formed SAMs is reported elsewhere (see Hein et al. Chem. Sci. 2021, 12, 2433-2440).

### S1.4: Voltammetric Measurements

Square wave voltammetry (SWV) measurements were conducted between −0.1 to 0.45 V with a step potential of 2 mV, amplitude of 20 mV and frequency of 25 Hz and were used to determine the E_{1/2} of 1.XB_{SAM} prior to capacitive measurements. Comparative continuous flow SWV measurements were carried out as previously reported (see Patrick et al. ChemRxiv 2021, DOI: 10.33774/chemrxiv-2021-2nqwc).

### S1.5: Capacitive Measurements

The film capacitance, C was obtained by electrochemical impedance spectroscopy (EIS) (see Hein et al. Chem. Commun. 2019, 55, 4849-4852). Measurements were carried out between 100 kHz and 0.1 Hz over 40 frequencies (logarithmically stepped), using a 10 mV AC amplitude and no equilibrium time. The DC potential applied was the E_{1/2} of 1.XB_{SAM}, as determined by SWV (see above), unless otherwise stated. The redox capacitance, Cᵣ was determined from the diameter of the semicircle of the capacitive Nyquist plot (approximated as C′ at the inflection point), and the corresponding frequency at this capacitance was denoted as fᵣ (typically 4-10 Hz). Density of state (DOS) experiments were performed at fixed frequency, fᵣ over a potential range of-0.1 to 0.4 V, with an equilibrium time of 1 s. Continuous capacitive measurements were performed with a time scan at fixed potential (E_{1/2}, unless otherwise stated) and fixed frequency (fᵣ, unless otherwise stated).

### S1.6: Continuous Capacitive Measurements: Pseudo-Flow Set-Up

A standard three-electrode cell (as described above in Section S1.2), filled with 4 mL of electrolyte (ACN/H₂O 99:1, 100 mM TBAClO₄, 10 mM HClO₄) was continuously stirred with a small stirrer bar. A syringe pump (Harvard Apparatus PHD 2000 Infuse/Withdraw) with PEEK tubing (Cole-Parmer) was used to inject 4 mL of the analyte solution (100 mM TBAX where X═target anion in the same solvent as above) at a flow rate of 800 μL min⁻¹ (injection duration is 5 min), leading to a final anion concentration, [A⁻]]=50 mM. Continuous capacitive measurements at a fixed frequency were performed as stated above in Section S1.5.

### S1.7: Continuous Capacitive Measurements: Continuous Flow Set-Up

A 3D-printed microfluidic cell (see Section S1.8) was utilised for continuous flow experiments. Electrolyte (ACN/H₂O 99:1, 100 mM TBAClO₄, 10 mM HClO₄) was continuously pushed through the cell with a syringe pump at a flow rate of 500 μL min⁻¹ (unless otherwise stated). Integrated into the flow line was an injector system (Rheodyne® Model 9725) through which the analyte solutions (of identical overall ionic strength, and acid concentration in the same solvent) were injected into the continuous flow. The majority of tubing used was Cole-Parmer PEEK tubing other than a small connecting section from the main tubing line to the cell, which was Cole-Parmer MasterFlex Peristaltic tubing.

### S1.8: 3D-Printed Microfluidic Cells

All microfluidic cells were produced with an Elegoo Mars 3D printer or a FormLabs Form 2 3D printer using FormLabs Tough 2000 resin, which is chemically resistant to a range of organic solvents. General designs were produced with Autodesk Fusion 360 (see FIG. 16E for annotated blueprint of design), which were then rendered into compatible (printable) designs in CHITUBOX. A Pt wire (counter electrode) was held in place in a channel adjacent to the main chamber with epoxy resin (Araldite Rapid), and the non-aqueous Ag|AgNO₃ reference electrode and Au disc electrode functionalised with 1.XB were inserted before each experiment, giving an airtight cell with an approximate chamber volume of 100 μL.

### S2—DATA ANALYSIS

### S2.1: Continuous Flow Sensogram Analysis

Continuous capacitive responses from measurements under flow were corrected for baseline degradation by linear scaling of all data points to the initial baseline recorded before the addition of the first anion aliquot. All sensograms represent the raw data without baseline corrections, but are in some cases offset in the x-axis (time domain) for illustrative purposes (in order to reduce overlap between sensograms obtained at different potentials). All sensing isotherms are baseline corrected and all quantitative analyses were carried out on baseline-corrected isotherms.

### S2.2: Binding Isotherm Analysis

All data analysis and binding isotherm fitting was carried out with OriginPro 2017. All binding constants are rounded to three significant figures and were obtained by fitting of the sensing isotherms to the Langmuir-Freundlich model (Eqn. 1; note when n=1, Eqn 1 simplifies to the standard Langmuir model), see Section S6 for further discussions. For quantitative analysis, the sensing isotherms for H₂PO₄⁻ were corrected for full protonation of H₂PO₄⁻ by the acidified electrolyte, as discussed in more detail in Section S9).

$$θ = \frac{{( {K}_{app} * [ {A}^{-} ] )}^{n}}{1 + {( {K}_{app} * [ {A}^{-} ] )}^{n}} \qquad Eqn . 1$$

Relative responses (in %) were calculated according to Cᵣₑₗ=(C_{A}_{^{−}}−C₀)/C₀×100.

### S2.3: Limit of Detection (LOD) and Sensitivity Determination

LODs were calculated according to a standard equation given by Eqn. 2 where o is the standard deviation of the baseline/blank and S is the slope of the linear region of the sensor response (herein also referred to as “sensitivity”). For continuous capacitive or voltammetric (see Patrick et al. ChemRxiv 2021, DOI: 10.33774/chemrxiv-2021-2nqwc) measurements, σ was determined from the root-mean-square deviation of a linear fit of 10 data points in the initial baseline of the sensograms, immediately preceding the response of the first addition. S was determined from the slope of a linear fit to the linear regime of each respective binding isotherm, with a linear range between 0-4.8 mM for HSO₄⁻, and either 11-15 mM (at E_{1/2}) or 11-20 mM (at E₋₂₀₀) for H₂PO₄⁻ (see Sections S6 and S9 for further discussions).

$$L O D = \frac{3 σ}{S} \qquad Eqn . 2$$

### S3: DOS REDOX CAPACITANCE

The redox capacitance of the SAM is inherently related to the redox activity of the interface. Specifically, the redox capacitance Cᵣ at a given potential is directly proportional to the redox density of states gr, i.e. Cᵣ(E)˜gᵣ(E) (see Lehr, J et al. Anal. Chem. 2014, 86, 2559-2564). In a generalized form, Cᵣ of a redox active SAM is then given by the following equation:

$${C}_{r} = \frac{Γ 4 {k}_{B} T}{e F} \qquad Eqn . 3$$

where Γ is the molecular surface coverage of redox active molecules (Γ=(1.01±0.18)×10⁻¹⁰ mol cm⁻² for 1.XB_{SAM}) (see Hein et al. Chem. Sci. 2021, 12, 2433-2440) and all other parameters are standard constants. A decrease in redox activity (i.e. a decrease in Γ/gᵣ) is thus directly reflected in a decreased redox capacitance. Thus, any voltammetric film instability will directly translate to an instability of the baseline Cᵣ. This can be avoided by using stable films, mitigated by acidification of the electrolyte, and/or can be corrected for.

As expected, the redox capacitance is significantly larger than the non-Faradaic capacitance, obtained at potentials at which no redox activity can be observed (here at −240 mV with respect to E_{1/2}, see FIG. 2A).

As shown in FIG. 4A and FIG. 2B, the redox density of states distribution shifts cathodically upon exposure to increasing concentrations of anions. It should be noted that the raw, absolute redox distributions, shown in FIG. 2B, display a significant peak suppression effect. This can be largely attributed to signal loss, i.e. non-ideal redox reversibility. Upon washing and measurements in fresh electrolyte some signal recovery can be observed (FIG. 2B, stars). Therefore, we hypothesise that some of the peak suppression observed may arise as a result of target binding, i.e. anion binding not only shifts the DOS distribution but may also change its shape (e.g. “suppress” the DOS).

Of note is that the signal degradation is relatively large in these DOS measurements, much larger than that observed under continuous flow or under pseudo flow at a fixed potential (see for example FIG. 8, FIG. 9 and FIG. 5A). This most likely arises from the comparably longer measurement times and the long times at very anodic potentials, at which point the interface is fully oxidized and more prone to degradation.

### S4: PSEUDO-FLOW CONTINUOUS CAPACITANCE

Continuous redox capacitance measurements under pseudo-flow (in a stirred, but otherwise static three-electrode cell) were carried out as detailed in Section S1.6. Simple, sequential anion titrations (addition of aliquots) can be performed via this methodology which are analogous to static titrations but allow for continuous monitoring of the induced response at each analyte concentration. As expected, the initially stable C_{E1/2} immediately dropped and quickly stabilised at an increasingly lower value upon addition of higher concentrations of HSO₄⁻ (FIG. 5A, trace with circular data points), affording response isotherms that are identical to those obtained under either fully static or continuous flow conditions (FIG. 5B). As expected, addition of PF₆⁻ (control) did not induce a significant capacitance response.

This pseudo-flow methodology was improved upon by continuous anion addition via syringe pump, herein over a time period of 5 min, see trace with square data points in FIG. 5A. This affords continuous sensing isotherms that are identical to those obtained by sequential addition of anion aliquots, however with a significantly larger number of data points (FIG. 5B, trace with triangular data points).

This presents a particularly useful, fast and experimentally straight-forward way of obtaining detailed information about interfacial binding events, an analysis that cannot be easily carried out by standard voltammetric methodologies.

The continuous addition, pseudo-flow approach can also aid in elucidating more complex response patterns, such as those observed at different electrode potentials (FIG. 5C). This is particularly useful at potentials that lie between E_{1/2} and the potential to which the redox distribution shifts to upon anion binding (here E₋₁₀₅ at 50 mM HSO₄). At these intermediate potentials, the response is characterised by an initial capacitance increase when the distribution shifts “into” the applied potential followed by a continuous monotonic decreasing response once the distribution shifts further cathodically (for example, see the response isotherms at E₋₅₀ or E₋₁₀₀).

### S5: CONTINUOUS CAPACITANCE UNDER FLOW

See FIG. 7.

### S6: BINDING ISOTHERM DATA

We initially fitted the response isotherms to the standard Langmuir model (Eqn. 1 with n=1), with only moderately good agreement. Perhaps initially surprising, this is actually expected on account of 1) protonation equilibria: While HSO₄⁻ is a weak conjugate base, some degree of protonation by the strong acid HClO₄ is still expected to occur (in particular at [A⁻]< [H⁺], i.e. below 10 mM anion), thereby “removing” some of the bisulfate anion from the equilibrium. Especially at low anion concentration, the sensor response is thus somewhat suppressed (this is also observed in the standard voltammetric format as detailed previously in Patrick et al. ChemRxiv (Analytical Chemistry) 30th August 2021, DOI: 10.33774/chemrxiv-2021-2nqwc). And 2) the potential-dependence of [Ox]/[Red] receptor distribution which in turn affects the overall anion binding strength, which changes during the course of the titration.

$$θ = \frac{{( {K}_{app} * [ {A}^{-} ] )}^{n}}{1 + {( {K}_{app} * [ {A}^{-} ] )}^{n}} \qquad Eqn . 1$$

Of note is that Kₐₚₚ represents an apparent binding constant and neither reflects anion binding to the oxidised receptor (K_{Ox}) nor the native, neutral receptor (K_{Red}), but can be interpreted as a general measure of sensor performance and anion binding strength. Similarly, no specific physico-chemical interpretation can be assigned to “n”, but it is typically interpreted as a measure of non-ideality/inhomogeneity, herein arising from partial protonation of the anions at low anion concentrations as well as the self-amplification effect.

Collated in the following tables (Table S6.1-S6.4) are all relevant parameters from analysis/fitting of individual continuous flow binding isotherms for HSO₄⁻ at different potentials, with a summary provided in Table S6.5. The equivalent data for H₂PO₄⁻ sensing is shown in Section S9.

TABLE S6.1
Apparent binding constants Kₐₚₚ (M⁻¹), maximum relative response
ΔCₘₐₓ (μF cm⁻²) and the index of heterogeneity n of 1.XBSAM towards
HSO₄⁻ obtained by fitting of the response isotherms (FIG. 9) at
E_{1/2} according to the Langmuir-Freundlich model (Eqn. 1). The
limit of detection (LOD, μM) was obtained by analysis of the
pseudo-linear region at low concentration (0-4.8 mM).

| | Kₐₚₚ | | ΔCₘₐₓ (μF | | LOD |
|---|---|---|---|---|---|
| Repeat | (M⁻¹) | n | cm⁻²) | R² | (μM) |
| 1 | 115 ± 10 | 2.02 ± 0.31 | − 42.0 ± 1.9 | 0.994 | 59.5 |
| 2 | 160 ± 14 | 1.71 ± 0.22 | −44.2 ± 1.6 | 0.995 | 25.4 |
| 3 | 112 ± 6 | 2.16 ± 0.22 | −45.4 ± 1.3 | 0.997 | 28.6 |
| 4 | 129 ± 9 | 2.21 ± 0.28 | −44.0 ± 1.4 | 0.996 | 45.8 |
| 5 | 104 ± 4 | 2.43 ± 0.18 | −45.9 ± 0.9 | 0.999 | 63.4 |
| Average | 121 ± 8 | 2.06 ± 0.24 | −44.4 ± 1.4 | 0.996 | 44.5 ± 15.5 |

TABLE S6.2
Apparent binding constants Kₐₚₚ (M⁻¹), maximum relative response
ΔCₘₐₓ (μF cm⁻²) and the index of heterogeneity n of 1.XB_{SAM} towards
HSO₄⁻ obtained by fitting of the response isotherms (FIG. 9) at
E₋₁₀₅ according to the Langmuir-Freundlich model (Eqn. 1).
The limit of detection (LOD, μM) was obtained by analysis
of the pseudo-linear region at low concentration (0-4.8 mM).

| | | | ΔCₘₐₓ (μF | | LOD |
|---|---|---|---|---|---|
| Repeat | Kₐₚₚ (M⁻¹) | n | cm⁻²) | R² | (μM) |
| 1 | 73.2 ± 4.4 | 2.41 ± 0.31 | 39.7 ± 1.6 | 0.996 | 83.1 |
| 2 | 47.3 ± 8.2 | 2.24 ± 0.55 | 46.6 ± 6.4 | 0.987 | 63.1 |
| 3 | 60.3 ± 5.5 | 1.84 ± 0.21 | 52.8 ± 3.2 | 0.997 | 89.6 |
| 4 | 74.4 ± 6.7 | 2.34 ± 0.43 | 44.8 ± 2.7 | 0.992 | 77.8 |
| Average | 62.7 ± 5.9 | 2.06 ± 0.30 | 46.0 ± 3.0 | 0.995 | 78.4 ± 9.8 |

TABLE S6.3
Apparent binding constants Kₐₚₚ (M⁻¹), maximum relative response
ΔCₘₐₓ (μF cm⁻²) and the index of heterogeneity n of 1.XB_{SAM} towards
HSO₄⁻ obtained by fitting of the response isotherms (FIG. 9) at
E₋₂₀₀ according to the Langmuir-Freundlich model (Eqn. 1).
The limit of detection (LOD, μM) was obtained by analysis
of the pseudo-linear region at low concentration (0-4.8 mM).

| | | | ΔCₘₐₓ (μF | | LOD |
|---|---|---|---|---|---|
| Repeat | Kₐₚₚ (M⁻¹) | n | cm⁻²) | R² | (μM) |
| 1 | 30.0 ± 6.2 | 1.22 ± 0.10 | 10.9 ± 1.3 | 0.999 | 59.7 |
| 2 | 37.8 ± 5.1 | 1.61 ± 0.15 | 8.15 ± 0.75 | 0.998 | 452 |
| 3 | 42.6 ± 4.7 | 1.66 ± 0.15 | 7.79 ± 0.57 | 0.998 | 120 |
| Average | 36.2 ± 4.1 | 1.45 ± 0.10 | 8.95 ± 0.64 | 0.999 | 78.9 ± 29.1 |

TABLE S6.4
Apparent binding constants Kₐₚₚ (M⁻¹), maximum relative response
ΔCₘₐₓ (μF cm⁻²) and the index of heterogeneity n of 1.XB_{SAM} towards
HSO₄⁻ obtained by fitting of the response isotherms (FIG. 9) at
E₋₁₀₀ according to the Langmuir-Freundlich model (Eqn. 1).
The limit of detection (LOD, μM) was obtained by analysis
of the pseudo-linear region at low concentration (0-4.8 mM).

| | | | ΔCmax (μF | | LOD |
|---|---|---|---|---|---|
| Repeat | Kapp (M⁻¹) | n | cm⁻²) | R² | (μM) |
| 1 | 257 ± 56 | 1.12 ± 0.21 | −7.19 ± 0.50 | 0.987 | 45.0 |
| 2 | 372 ± 67 | 1.42 ± 0.30 | −5.95 ± 0.31 | 0.983 | 36.3 |
| 3 | 338 ± 72 | 1.20 ± 0.25 | −5.23 ± 0.34 | 0.983 | 41.6 |
| Average | 320 ± 62 | 1.23 ± 0.24 | −6.09 ± 0.36 | 0.985 | 40.9 ± 4.3 |

TABLE S6.5
Apparent binding constants Kₐₚₚ (M⁻¹) and maximum relative response Δ
Cₘₐₓ (μF cm⁻²) of 1.XB^{SAM} towards HSO₄⁻ obtained by fitting of the
response isotherms (FIG. 9) at different potentials according to the Langmuir-Freundlich
model (LFM, Eqn. 1). The limit of detection (LOD, μM) and sensitivity (μF
mM⁻¹) was obtained by analysis of the pseudo-linear region at low concentration
(0-4.8 mM, see S2.3). The sensor performance in an optimised voltammetric format
(SWV) is also shown for comparison (see Patrick et al. ChemRxiv (Analytical
Chemistry) 30th Aug. 2021, DOI: 10.33774/chemrxiv-2021-2nqwc) N.a.—not applicable.

| | Redox capacitance | | | | Voltammetry^{a} |
|---|---|---|---|---|---|
| Edc | E ± 100^{a} | E_{1/2}^{b} | E₋₁₀₅^{c} | E₋₂₀₀^{d} | n.a. |
| LOD (μM) | 42.4 ± 4.0 | 44.5 ± 15.5 | 70.4 ± 18.2 | 78.9 ± 29.1 | 59.8 ± 5.5^{c} |
| Sensitivity | 0.63 ± 0.10 | 2.06 ± 0.60 | 1.03 ± 0.17 | 0.14 ± 0.03 | 3.36 ± 1.22^{h} |
| (μF mM⁻¹) | | | | | |
| Kₐₚₚ (M⁻¹) | 320 ± 60 | 121 ± 6 | 64.2 ± 7.7 | 36.2 ± 7.2 | 68.6 ± 17.6 |
| ΔCₘₐₓ (μF cm⁻²)^{f} | −6.09 ± 0.35 | −44.4 ± 1.0 | 46.0 ± 3.05 | 8.95 ± 1.12 | −116 ± 9^{g} |

Errors represent one standard deviation of: ^{a}4, ^{b}5, ^{c}6, ^{d}3, independent repeats.
^{e}under highly optimised conditions with advanced data processing/fitting. Without this
optimised analysis the LOD is significantly worse (825 ± 138 μM) (see Patrick et al.
ChemRxiv (Analytical Chemistry) 30th Aug. 2021, DOI: 10.33774/chemrxiv-2021-2nqwc);
^{f}at [HSO₄⁻] = ∞.
^{g}in mV · h − in mV mM⁻¹.

Not only does the voltammetric approach require significant data analysis/processing (herein via a custom-written data analysis script that can extract the peak-potential from the raw voltammograms via peak fitting), but it is also associated with significantly lower temporal resolution (≈5.5 s) and higher baseline degradation: specifically, without improved data processing/analysis by peak fitting the LOD for the continuous SWV methodology is even worse (825±138 μM; 18.5-fold larger than that attained by redox capacitance measurements (see Patrick et al. ChemRxiv (Analytical Chemistry) 30th August 2021, DOI: 10.33774/chemrxiv-2021-2nqwc).

### S7: THEORETICAL CONSIDERATIONS OF BINDING CONSTANTS AT DIFFERENT POTENTIALS DERIVATION OF K_{THEO}

Starting from the Nernst equation (Eqn. S7.1), an approximation of

$$- \frac{R T}{n F} = 59$$

mV is first made to simplify Eqn. S7.1 to Eqn. S7.2, which can be rearranged to give Eqn. S7.3.

$$Δ E = - \frac{R T}{n F} ln ( \frac{Ox}{Red} ) \qquad ( Eqn . S7 \text{.1} )$$

$$Δ E = - 59 mV * log ( \frac{Ox}{Red} ) \qquad ( Eqn . S7 \text{.2} )$$

$$\frac{Ox}{Red} = 10 \frac{Δ E}{- 0.059 mV} \qquad ( Eqn . S7 \text{.3} )$$

The theoretical, average binding constant K_{Theo} is defined by Eqn. S7.4, as the weighted mean of the binding constants to the oxidized and reduced interfaces, given by K_{Ox} and K_{red} respectively, and the respective molar fractions of oxidized and reduced receptor states. K_{Theo} can alternatively be described by Eqn. S7.7, where the proportions of oxidized and reduced receptors at the interface have been replaced with the variables % Red and % 0x (eqn. S7.5 and S7.6).

$${K}_{T h e o} = {K}_{O x} * \frac{Ox}{Red + Ox} + {K}_{r e d} * \frac{Red}{R e d + O x} \qquad ( Eqn . S7 \text{.4} )$$

$$\% Red = \frac{Red}{Ox + Red} \qquad ( Eqn . S7 \text{.5} )$$

$$\% Ox = \frac{Ox}{Ox + Red} \qquad ( Eqn . S7 \text{.6} )$$

$${K}_{T h e o} = {K}_{O x} * \% Ox + {K}_{r e d} * \% Red \qquad ( Eqn . S7 \text{.7} )$$

As % Ox and % Red add up to 1, % Ox can be substituted for (1−% Red) in Eqn. S7.7, affording Eqn. S7.8.

$${K}_{Theo} = {K}_{Ox} * ( 1 - \% Red ) + {K}_{red} * \% Red \qquad ( Eqn . S7 \text{.8} )$$

Eqn. S7.5 can be rearranged as shown in the steps described by Eqns. S7.9-S7.11. Substitution of Eqn. S7.3 into Eqn. S7.11 then affords Eqn. S7.12.

$$\frac{1}{\% Red} = \frac{O x + R e d}{R e d} \qquad ( Eqn . S7 \text{.9} )$$

$$\frac{1}{\% Red} = \frac{O x}{R e d} + \frac{R e d}{R e d} = \frac{O x}{R e d} + 1 \qquad ( Eqn . S7 \text{.10} )$$

$$\% Red = {( \frac{Ox}{Red} + 1 )}^{- 1} \qquad ( Eqn . S7 \text{.11} )$$

$$\% Red = {( 10 \frac{Δ E}{- 0.059} + 1 )}^{- 1} \qquad ( Eqn . S7 \text{.12} )$$

Eqn. S7.13 is afforded by substituting Eqn. S7.12 into Eqn. S7.8, and undergoes final simplifications to result in Eqn. S7.15 which correlates K_{Theo} to the applied potential (AE with respect to E_{1/2}), as shown in FIG. 10. The upper and lower bounds are K_{Ox}=1590 M⁻¹ and K_{Red}=20 M⁻¹ which were obtained as described previously (Hein et al. Chem. Sci. 2021, 12, 2433-2440).

$${K}_{T h e o} = {K}_{O x} * ( 1 - {( 10 \frac{Δ E}{- 0.059} + 1 )}^{- 1} ) + {K}_{r e d} * {( 10 \frac{Δ E}{- 0.059} + 1 )}^{- 1} \qquad ( Eqn . S7 \text{.13} )$$

$${K}_{T h e o} = {K}_{O x} - {K}_{O x} {( 10 \frac{Δ E}{- 0.059} + 1 )}^{- 1} + {K}_{r e d} * {( 10 \frac{Δ E}{- 0.059} + 1 )}^{- 1} \qquad ( Eqn . S7 \text{.14} )$$

$${K}_{T h e o} = {K}_{O x} + ( {K}_{R e d} - {K}_{O x} ) {( 10 \frac{Δ E}{- 0.059} + 1 )}^{- 1} \qquad ( Eqn . S7 \text{.15} )$$

Of note is that K_{Theo} and Kₐₚₚ are related, but not identical. Firstly, K_{Theo} was calculated based on K_{Ox}=1590 M⁻¹ and K_{Red}=20 M⁻¹ as obtained from voltametric isotherm analysis in the presence of only 100 μM acid (Hein et al. Chem. Sci. 2021, 12, 2433-2440). However, herein, the higher acid concentration of 10 mM induces some degree of protonation of HSO₄⁻, such that binding is somewhat attenuated. Secondly, the experimentally determined Kₐₚₚ represents an overall sensor performance over a range of anion concentrations. As a result of the self-amplification effect discussed in the elsewhere (FIG. 11), the ratio of Ox/Red and thus K_{Theo} is dependent on the anion concentration, such that K_{Theo} is larger in the presence of higher concentrations of anions (as higher [A⁻] shift the redox distribution further cathodically, such that at the constant initial electrode polarization the ratio of Ox/Red rises). Kₐₚₚ thus “spans” a range of different K_{Theo}. For example, for measurements at E_{1/2} binding is switched on from an initial theoretical binding constant of K_{Theo, E1/2}=805 M⁻¹ to a maximum of K_{Ox}=1580 M⁻¹ (FIG. 10).

In contrast, during standard voltammetric analysis no such “self-amplification” is observed and the overall, apparent binding remains constant (at least as transduced as the shift in half-wave potential).

### S8: MEASUREMENTS AT DIFFERENT POTENTIALS

Modulation of the sensing performance according to the principles of changing electrode potentials, can be carried out across a wide range of electrode potential, from approximately 118 mV anodic of E_{1/2} (corresponding to a 99:1 ratio Ox/Red redox states such that a residual redox capacitance can be resolved) and 118 mV+ΔE_{max, binding} in the cathodic direction (here ≈220 mV for HSO₄⁻ and ≈310 mV for H₂PO₄⁻).

By judicious choice of the applied electrode potential the sensitivity and/or limit of detection (LOD) of the sensor can thus be tuned as desired, for example to obtain a threshold sensing response to any precise analyte concentration. Specifically, at sufficiently cathodic potentials, low anion concentrations do not induce sufficient voltammetric shifts of the DOS to induce significant Faradaic activity (i.e. the measured capacitance remains (largely) non-Faradaic, see the isotherm at E₋₂₀₀, FIG. 9).

### S9: REDOX CAPACITIVE SENSING OF OTHER ANIONS

### S9.1: Sensing of Phosphate

As shown in FIGS. 13A and 13B, H₂PO₄⁻ is completely unresponsive at concentrations below 10 mM. This can be attributed to full protonation of this more basic anion by the acidic electrolyte (Patrick et al. ChemRxiv (Analytical Chemistry) 30th August 2021, DOI: 10.33774/chemrxiv-2021-2nqwc). In order to account for the complete “removal” of H₂PO₄⁻ from the equilibrium, the corrected isotherms in FIG. 13C were thus corrected by omission of all data points between 0-10 mM and correction of all other data points by −10 mM [H₂PO₄⁻]. From these corrected isotherms the apparent H₂PO₄⁻ binding constants, as stated elsewhere and the subsequent tables, were obtained. Similarly, the LODs were calculated based on the corrected isotherms, and in effect represent the LODs that are underestimated by 10 mM (Tables S9.1-S9.3).

TABLE S9.1
Apparent binding constants Kₐₚₚ (M⁻¹), maximum relative
response ΔCₘₐₓ (μF cm⁻²) and the index of heterogeneity n of 1.XB_{SAM}
towards H₂PO₄⁻ obtained by fitting of the response isotherms
(FIG. 13A) at E_{1/2} according to the Langmuir-Freundlich
model (LFM, Eqn. 1). The limit of detection (LOD, μM)
and sensitivity were obtained by analysis of the pseudo-
linear region at low concentration (11-15 mM).

| | | | ΔCₘₐₓ (μF | | LOD |
|---|---|---|---|---|---|
| Repeat | Kapp (M⁻¹ ) | n | cm⁻²) | R² | μM |
| 1 | 406 ± 31 | 3.13 ± 0.32 | −53.1 ± 0.8 | 0.999 | 6.95 |
| 2 | 355 ± 2 | 3.13 ± 0.03 | −53.0 ± 0.1 | 0.999 | 5.43 |
| 3 | 440 ± 11 | 4.53 ± 0.16 | −71.3 ± 0.2 | 0.999 | 6.02 |
| Average | 398 ± 14 | 3.52 ± 0.17 | −59.1 ± 0.4 | 0.999 | 6.13 |

TABLE S9.2
Apparent binding constants Kₐₚₚ (M⁻¹), maximum relative
response ΔCₘₐₓ (μF cm²) and the index of heterogeneity n of
1.XB_{SAM} towards H₂PO₄⁻ obtained by fitting of the response isotherms
(FIG. 13B) at E₋₂₀₀ according to the Langmuir-Freundlich model
(LFM, Eqn. 1). The limit of detection (LOD, μM) and sensitivity
were obtained by analysis of the pseudo-linear region at low
concentration (11-20 mM).

| | | | ΔCₘₐₓ (μF | | LOD |
|---|---|---|---|---|---|
| Repeat | Kₐₚₚ (M⁻¹ ) | n | cm⁻²) | R² | μM |
| 1 | 93.8 ± 3.9 | 2.99 ± 0.30 | 53.1 ± 2.3 | 0.999 | 8.69 |
| 2 | 139 ± 3 | 3.42 ± 0.20 | 48.6 ± 0.8 | 0.999 | 1.28 |
| 3 | 163 ± 9 | 6.06 ± 1.40 | 43.1 ± 1.2 | 0.997 | 2.21 |
| Average | 130 ± 12 | 3.41 ± 0.61 | 47.6 ± 2.9 | 0.998 | 4.87 |

TABLE S9.3
Apparent binding constants Kₐₚₚ (M⁻¹) and maximum relative response
ΔCₘₐₓ (μF cm²) of 1.XB_{SAM} towards H₂O₄⁻ and H₂PO₄⁻ obtained
by fitting of the response isotherms (FIGS. 7 and 13C, respectively) at E₋₂₀₀
and E_{1/2} according to the Langmuir-Freundlich model (LFM, Eqn. 1). The
limit of detection (LOD, μM) and sensitivity (μF mM⁻¹)
was obtained by analysis of the pseudo-linear region at low concentration
(see below and S2.3).

| | Redox Capacitance HSO₄⁻ | | Redox Capacitance H₂ PO₄⁻ | |
|---|---|---|---|---|
| Edc | E₋₂₀₀ ^{a} | E_{1/2} ^{b} | E₋₂₀₀ ^{a} | E_{1/2} ^{a} |
| LOD μM | 78.9 ± 29.1 | 44.5 ± 15.5 | 4.06 ± 3.29^{c} | 6.14 ± 0.63^{d} |
| Sensitivity (μF mM⁻¹) | 0.14 ± 0.03 | 2.06 ± 0.60 | 3.75 ± 0.85^{c} | 13.2 ± 2.81^{d} |
| Kₐₚₚ (M⁻¹) | 36.2 ± 7.2 | 121 ± 6 | 132 ± 29 | 401 ± 35 |
| ΔCₘₐₓ (μF cm⁻²)^{e} | 8.95 ± 1.12 | −44.4 ± 1.0 | 48.3 ± 4.1 | −59.1 ± 8.6 |

^{a}3 ^{b}5 independent repeats.
Obtained from a linear range of ^{c}11-20 mM H₂PO₄⁻ or
^{d}11-15 mM H₂PO₄⁻.
^{e}at [A⁻] = ∞.

The slightly lower LOD for H₂PO₄⁻ at E₋₂₀₀ in comparison to E_{1/2} arises from a lower standard deviation of the baseline at E₋₂₀₀ (i.e. the non-Faradaic capacitive baseline is more stable than the Faradaic one). As shown in Table S9.3, the sensitivity and Kₐₚₚ are significantly higher at E_{1/2}. Of further note is that the accuracy of the slopes of the linear range (sensitivities) and LODs is lower for H₂PO₄⁻ (in comparison to HSO₄⁻), as fewer data points fall within the linear sensor response regime.

It should be noted that the standard V_{Sample}=0.5 mL was insufficient to observe the full extent of the response to Cl⁻ (i.e. the response to each analyte injection did not plateau), thus V_{Sample} was increased to 1 mL, doubling the duration of time of exposure of Cl-to the interface. This was shown to be sufficient to observe an equilibrium response for concentrations of Cl⁻ up to 20 mM, but a notable sharpening of the peaks with increasing [Cl⁻], prevented investigation of concentrations above 20 mM (FIG. 14).

As shown in FIG. 14, Cl⁻ induces a response that is also characterised by a significantly slower baseline recovery than observed for the oxoanions, an observation potentially arising from slow anion decomplexation. Interestingly, slower anion decomplexation is observed at the more cathodic potential.

### S10: MEASUREMENTS AT MULTIPLE POTENTIALS AND FREQUENCIES

As alluded to elsewhere, the redox capacitive methodology is further tuneable by adjusting a wide variety of other measuring parameters. This includes, but is not limited to measurements at different frequencies, AC amplitudes and at multiple different potentials of frequencies.

For example, as shown in FIG. 15A, measurements can be carried out at two (or more) different potentials. This was achieved by alternately recording measurements at the two different potentials (i.e. fast switching back and forth), which is associated with a slightly worse temporal resolution but affords double the information density within the same experiment/time span. As expected, the sensograms and isotherms obtained in this manner (FIG. 15B) are completely identical to those obtained under “standard” conditions.

We also envision that this two-potential approach can be employed for ratiometric sensing. Similarly, measurements can be carried out at multiple different AC frequencies, as shown for pseudo-flow sensing in FIG. 15C. In analogy to the two-potential approach this was herein achieved by fast switching between the different frequencies, however overtone frequencies, can, depending on the hardware, be obtained simultaneously (i.e. truly simultaneously and without loss of temporal resolution). This again affords a higher information density and can potentially be used to further tune the sensor performance.

### S11 REFERENCES

All publications referred to in the present disclosure are herein incorporated by reference in their entirety, including but not limited to the following:
1. Hein et al. Chem. Sci. 2021, 12, 2433-2440.
2. Patrick et al. Chem. Eur. J. 2021, 27, 10201-10209.
3. Patrick et al. ChemRxiv (Analytical Chemistry) 30th August 2021, DOI: 10.33774/chemrxiv-2021-2nqwc (accessed 2021 Oct. 15).
4. Hein et al. Chem. Commun. 2019, 55, 4849-4852.
5. Lehr, J et al. Anal. Chem. 2014, 86, 2559-2564.

1-24. (canceled)

25. An electrochemical method of sensing target particles in a carrier medium under flow, which method comprises:
(A) providing a flow path that comprises an electrode, the electrode being positioned to contact the carrier medium when the carrier medium flows along the flow path, and the electrode comprising redox-active receptors capable of binding to the target particles to change the redox capacitance of the electrode;
(B) flowing the carrier medium along the flow path over a flow period, such that a continuously changing portion of the carrier medium contacts the electrode throughout the flow period; and
(C) continuously obtaining redox capacitance measurements of the electrode over the flow period, thereby sensing the binding of target particles to the redox-active receptors and hence the presence, or absence, of target particles in the portion of carrier medium that is in contact with the electrode at each time point over the flow period.

26. The method of claim 25, wherein the target particles are target ions.

27. The method of claim 26, wherein the target ions are anions.

28. The method of claim 27, wherein the target ions are selected from Cl⁻, F⁻, Br⁻, I⁻, HSO₄⁻, H₂PO₄⁻, TcO₄⁻ and NO₃⁻.

29. The method of claim 26, wherein the target ions are cations.

30. The method of claim 25, wherein the target particles are neutrally charged molecules or atoms.

31. The method of claim 25, wherein the carrier medium is aqueous.

32. The method of claim 31, wherein the carrier medium is water and the method is a method for sensing whether the target particles are present in the water.

33. The method of claim 25, wherein step (C) comprises determining the concentration of target particles in the portion of the carrier medium that is in contact with the electrode at each time point over the flow period.

34. The method of claim 25, wherein:
i) the flow period is at least 10 minutes; or
ii) the flow period is at least 30 minutes; or
iii) the flow period is at least 60 minutes; or
iv) the flow period is at least 3 hours.

35. The method of claim 25, wherein the flow path is comprised in a microfluidic cell.

36. The method of claim 25, wherein the redox active receptors comprise a redox active portion that is selected from: a ferrocene group, a group derived from methylene blue; a quinone; and a metallic chemical complex comprising a transition metal, wherein the transition metal is preferably Fe, Ru, Ti, V, Mn, Cr, Co, Ni, Nb, Mo or Os.

37. The method of claim 36, wherein the redox active receptors comprise a ferrocene group.

38. The method of claim 37, wherein the redox active receptors are ferrocene-isophthalamide-triazole self-assembled monolayers (SAMs) or ferrocene-isophthalamide-iodotriazole self-assembled monolayers (SAMs).

39. The method of claim 25, which further comprises acidifying the the carrier medium before or during the step of flowing the carrier medium along the flow path.

40. The method of claim 25, comprising obtaining redox capacitance measurements of the electrode in which the electrode potential is the E_{1/2} potential of the electrode.

41. The method of claim 25, comprising obtaining redox capacitance measurements of the electrode in which the electrode potential is different from the E_{1/2} potential of the electrode.

42. The method of claim 25, wherein the redox capacitance measurements of the electrode are obtained at a single electrode potential.

43. The method of claim 25, wherein the redox capacitance measurements of the electrode are obtained at two or more electrode potentials by cycling between the two or more electrode potentials over the flow period.

44. The method of claim 25, wherein the redox capacitance measurements of the electrode are obtained at a single frequency and/or a single AC amplitude.

45. The method of claim 25, wherein the redox capacitance measurements of the electrode are obtained at two or more frequencies by cycling between the two or more frequencies over the flow period and/or two or more AC amplitudes by cycling between the two or more AC amplitudes over the flow period.
